(19) United States
(12) Patent Application Publication
(10) Pub. No.: US 2025/0316335 A1
(43) Pub. Date: Oct. 9, 2025

(54) SYSTEMS, METHODS, AND MEDIA FOR CLASSIFYING GENETIC SEQUENCING RESULTS

(71) Applicant: Arc Bio, LLC, Cambridge, MA (US)

(72) Inventors: Alain WATTS, Scotts Valley, CA (US); Philip UREN, Scotts Valley, CA (US)

(21) Appl. No.: 18/865,188

(22) PCT Filed: May 12, 2023
(86) PCT No.: PCT/US2023/022099

§ 371 Date: Nov. 12, 2024

Related U.S. Application Data
(60) Provisional application No. 63/341,874, filed on May 13, 2022.
(60) Provisional application No. 63/407,971, filed on Sep. 19, 2022.

(51) Int. Cl. G16B 30/00; G06F 30/27; G16B 40/20

(52) U.S. Cl. CPC G16B 30/00; G06F 30/27; G16B 40/20

(57) ABSTRACT

In accordance with some embodiments, systems, methods, and media for classifying genetic sequencing results are provided. In some embodiments, a system includes a processor programmed to: receive a sample genetic sequencing result for a reference organism and for a host organism, generate a plurality of synthetic genetic sequencing results by combining a portion of the sample genetic sequencing result for the reference organism and the host organism, generate a matrix by cross-referencing a pair of synthetic genetic sequencing results, generate a model based on the synthetic genetic sequencing results, determine at least one threshold based on the matrix, update the model based on the threshold, receive a clinical sample genetic sequencing result, identify, utilizing the model, any values in the clinical sample genetic sequencing result that are likely to be diagnostically significant; generate a report; and cause the report to be presented to a user.

### CROSS-REFERENCE TO RELATED APPLICATIONS

This application is based on, claims the benefit of, and claims priority to U.S. Provisional Patent Application No. 63/341,874, filed May 13, 2022, and U.S. Provisional Patent Application No. 63/407,971, filed Sep. 19, 2022, each of which is hereby incorporated by reference herein in its entirety for all purposes.

### STATEMENT REGARDING FEDERALLY SPONSORED RESEARCH

N/A

### BACKGROUND

Genetic sequencing can identify genetic material present in a sample. This can be useful for identifying the sources of certain genetic material present in a sample, for example, identifying certain pathogens present in a sample. However, errors in identifying the source of certain genetic material can often occur. Thus, there is a need to more accurately identify the sources of certain genetic material present in a sample.

Accordingly, new systems, methods, and media for classifying genetic sequencing results are desirable.

### SUMMARY

In accordance with some embodiments of the disclosed subject matter, systems, methods, and media for classifying genetic sequencing results are provided.

In accordance with some embodiments of the disclosed subject matter, a system for classifying a genetic sequencing result for a sample is provided, the system having at least one hardware processor that is programmed to: receive a clinical sample genetic sequencing result for a clinical sample, wherein the clinical sample genetic sequencing result includes a plurality of values that are each indicative of a number of reads detected in the clinical sample for a respective reference organism of a plurality of reference organisms. The hardware processor is also programed to identify a value in the clinical sample genetic sequencing result that is over a detection threshold associated with an organism, and to determine, utilizing a model, that the value is unlikely to be diagnostically significant. The hardware processor is further programed to generate a report based on the clinical sample genetic sequencing result and any reference organisms associated with a value identified as likely to be diagnostically significant; and to cause at least a portion of the report to be presented to a user with an indication of any organisms associated with a value identified as likely to be diagnostically significant.

In some embodiments, the at least one hardware processor is further programmed to: generate a distribution for each of reference organisms in the plurality of reference organisms based on the plurality of sample genetic sequencing results, associate, for each of the plurality of reference organisms, a threshold that is based on the distribution; and to generate at least one matrix of replicate-averaged signal for each reference organism in the plurality of reference organisms by cross-referencing at least one synthetic genetic sequencing result for each reference organism with at least one other synthetic genetic sequencing result for said same reference organism. The hardware processor can be further programmed to update the threshold for each reference organism based on the matrix of replicate-averaged signal, and identify, utilizing the model, any values in the clinical sample genetic sequencing result that are likely to be diagnostically significant based on the threshold associated with each reference organism.

In some embodiments, the at least one hardware processor is further programmed to train a neural network using the plurality of synthetic genetic sequencing results, provide the clinical sample genetic sequencing result as input to the trained neural network, and receive, from the trained neural network, output identifying any values in the clinical sample genetic sequencing result that are likely to be diagnostically significant.

In some embodiments, the at least one hardware processor is further programmed to receive at least one sample genetic sequencing result for a reference organism corresponding to a respective reference organism sample, receive at least one sample genetic sequencing result for a host organism corresponding to a respective host organism sample, and to generate a plurality of synthetic genetic sequencing results corresponding to a respective plurality of synthetic samples each containing a combination of the host reference organism and the reference organism by combining at least a portion of the sample genetic sequencing result for the reference organism with at least a portion of the sample genetic sequencing result for the host organism for each synthetic sample. Each synthetic genetic sequencing result includes a plurality of values that are each indicative of a number of reads detected in the synthetic sample for a respective reference organism. The hardware processor can be further programmed to generate at least one matrix of replicate-averaged signal by cross-referencing at least one synthetic genetic sequencing result with at least one other synthetic genetic sequencing result, generate a model based on the at least one sample genetic sequencing result for a reference organism and the at least one sample genetic sequencing result for a host organism, determine at least one threshold based on the at least one matrix of replicate-averaged signal, and to update at least a portion of the model based on the at least one threshold.

In some embodiments, the at least one hardware processor is further programmed to (i) receive a plurality of sample genetic sequencing results for a plurality of reference organisms corresponding to a respective plurality of reference organism samples, (ii) generate a synthetic genetic sequencing result by combining at least a portion of a sample genetic sequencing result for a reference organism with at least a portion of the sample genetic sequencing result for the host organism; and (iii) repeat (ii) for each reference organism sample of the plurality of reference organism samples.

In some embodiments, the at least one hardware processor is further programmed to generate a sufficient number of synthetic genetic sequencing results such that the number of synthetic genetic sequencing results in the plurality of synthetic genetic sequencing results is at least 10× greater than the number of sample genetic sequencing results for reference organisms in the plurality of sample genetic sequencing results for a plurality of reference organisms.

In some embodiments, the at least one hardware processor is further programmed to determine at least one threshold based on the at least one matrix of replicate-averaged signal, using conditional probability.

In some embodiments, the at least one hardware processor is further programmed to determine at least one threshold based on the at least one matrix of replicate-averaged signal, using a combination of conditional probability and at least one loss function.

In accordance with some embodiments, a method for classifying a genetic sequencing result for a sample is provided, the method including: receiving a clinical sample genetic sequencing result for a clinical sample, the clinical sample genetic sequencing result including a plurality of values that are each indicative of a number of reads detected in the clinical sample for a respective reference organism of a plurality of reference organisms, identifying a value in the clinical sample genetic sequencing result that is over a detection threshold associated with an organism, determining, utilizing a model, that the value is unlikely to be diagnostically significant, generating a report based on the clinical sample genetic sequencing result and any reference organisms associated with a value identified as likely to be diagnostically significant; and, causing at least a portion of the report to be presented to a user with an indication of any organisms associated with a value identified as likely to be diagnostically significant.

In accordance with some embodiments of the disclosed subject matter, a non-transitory computer readable medium containing computer executable instructions that, when executed by a processor, cause the processor to perform a method for classifying a genetic sequencing result for a sample is provided, the method including: receiving a clinical sample genetic sequencing result for a clinical sample, the clinical sample genetic sequencing result including a plurality of values that are each indicative of a number of reads detected in the clinical sample for a respective reference organism of a plurality of reference organisms, identifying a value in the clinical sample genetic sequencing result that is over a detection threshold associated with an organism, determining, utilizing a model, that the value is unlikely to be diagnostically significant, generating a report based on the clinical sample genetic sequencing result and any reference organisms associated with a value identified as likely to be diagnostically significant; and, causing at least a portion of the report to be presented to a user with an indication of any organisms associated with a value identified as likely to be diagnostically significant.

In accordance with some embodiments of the disclosed subject matter, a system for classifying a genetic sequencing result for a sample is provided, the system comprising: at least one hardware processor that is programmed to: receive a clinical sample genetic sequencing result for a clinical sample, the clinical sample genetic sequencing result comprising a plurality of values that are each indicative of a number of reads detected in the clinical sample for a respective reference organism of a plurality of reference organisms; identify, for each of a plurality of members of a taxonomic level, a count of unique reads that align with only a single member of that taxonomic level; determine, for the taxonomic level, a homogeneity metric H indicative how high the unique read count of a member of the taxonomic level with a highest unique read count is compared to a member of the taxonomic level with a next highest unique read count; determine, for each of the plurality of members of the taxonomic level, a uniqueness metric U based on the count of unique reads associated with that member and the homogeneity metric associated with the taxonomic level; generate a report based on the clinical sample genetic sequencing result and the uniqueness metric associated with each of the plurality of members of the taxonomic level; and cause at least a portion of the report to be presented to a user with an indication of any organisms associated with a uniqueness value above a threshold.

In some embodiments, the plurality of members the taxonomic level correspond to different strains.

In some embodiments, wherein the plurality of members the taxonomic level correspond to different species.

In some embodiments, wherein the homogeneity metric is calculated using the following:

$$H = \frac{{R}^{m}}{{R}^{m} + {R}^{n}} ,$$

where R^{m} is the count of unique reads for the member with the highest count of unique reads, and Rⁿ is the count of unique reads for the member with the next highest count of unique reads.

In some embodiments, wherein the uniqueness metric is calculated using the following:

$$U = H \frac{{R}^{i}}{{R}^{m}} ,$$

where R^{m} is the count of unique reads for the member with the highest count of unique reads, and Rⁱ is the count of unique reads of the member for which U is being determined.

In some embodiments, wherein the at least one hardware processor that is programmed to: identify, for each of a plurality of members of a taxonomic level, the count of unique reads.

In accordance with some embodiments of the disclosed subject matter, a system for classifying a genetic sequencing result for a sample is provided, the system comprising: at least one hardware processor that is programmed to: receive a clinical sample genetic sequencing result for a clinical sample, the clinical sample genetic sequencing result comprising a plurality of values that are each indicative of a number of reads detected in the clinical sample for a respective reference organism of a plurality of reference organisms; identify a value in the clinical sample genetic sequencing result that is over a detection threshold associated with a member of a taxonomic level; determine, utilizing a model, that the value is unlikely to be diagnostically significant; identify, for each of a plurality of members of the taxonomic level, a count of unique reads that align with only a single member of that taxonomic level; determine, for the taxonomic level, a homogeneity metric H indicative how high the unique read count of a member of the taxonomic level with a highest unique read count is compared to a member of the taxonomic level with a next highest unique read count; determine, for each of the plurality of members of the taxonomic level, a uniqueness metric U based on the count of unique reads associated with that member and the homogeneity metric associated with the taxonomic level; generate a report based on the clinical sample genetic sequencing result, the uniqueness metric associated with each of the plurality of members of the taxonomic level, and any reference organisms associated with a value identified as likely to be diagnostically significant; and cause at least a portion of the report to be presented to a user with an indication of any organisms associated with a value identified as likely to be diagnostically significant.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1 shows an example of a system for classifying genetic sequencing results in accordance with some embodiments of the disclosed subject matter.

FIG. 2 shows an example of hardware that can be used to implement a computing device, and a server, shown in FIG. 1 in accordance with some embodiments of the disclosed subject matter.

FIG. 3 shows an example of a process for determining and/or optimizing pathogen-specific adaptive thresholds in accordance with some embodiments of the disclosed subject matter.

FIG. 4 shows an example of a process for generating synthetic sequence data and expanded libraries in accordance with some embodiments of the disclosed subject matter.

FIG. 5 shows an example of a Species×Species matrices of replicate-averaged signal with paired covariance matrices in accordance with some embodiments of the disclosed subject matter.

FIG. 6 shows a graphical representation of the relationship between LoB, LoD, and LoQ, with respect to measurand concentration.

FIG. 7 shows an example of a topology of an autoencoder that can be generated to predict pathogen-specific adaptive thresholds using mechanisms described herein in accordance with some embodiments of the disclosed subject matter.

FIG. 8 shows an example representation of a graph associated with a particular type of organism(s) with multiple taxonomic levels, and an indication of a number of reads from a sample that uniquely map to each taxa within a taxonomic level in accordance with some embodiments of the disclosed subject matter.

FIG. 9 shows an example representation of proportions of a unique reads that map to various taxa within a taxonomic level in accordance with some embodiments of the disclosed subject matter.

FIG. 10 shows an example of a process for determining and using a uniqueness metric to classify genetic sequencing results in accordance with some embodiments of the disclosed subject matter.

FIG. 11 shows an example of a process for using pathogen-specific adaptive thresholds and a uniqueness metric to classify genetic sequencing results in accordance with some embodiments of the disclosed subject matter.

FIGS. 12A and 12B show examples of how using pathogen-specific adaptive thresholds and/or a uniqueness metric can impact the precision and sensitivity of classification of genetic sequencing results in accordance with some embodiments of the disclosed subject matter.

### DETAILED DESCRIPTION

In accordance with various embodiments, mechanisms (which can, for example, include systems, methods, and media) for classifying genetic sequencing results are provided.

In accordance with some embodiments of the disclosed subject matter, mechanisms described herein can be used to generate a model that can used to classify results of genetic sequencing as more or less likely to be clinically significant. In general, a sample (e.g., blood, sputum, fecal matter, etc.) can be sequenced to attempt to identify organisms present in the sample. Next generation sequencing techniques can be used to identify reads relatively inexpensively and relatively quickly (e.g., on the order of dozens to thousands of base pairs in length) present in the sample. The reads can then be aligned to reference sequences for various organisms to attempt to identify which organism a particular read originated from.

Various sources of error can cause false positive results to be included in the aligned reads. A potential source of error stems from conserved sequences. In evolutionary biology, conserved sequences are sequences of nucleic acids (such as DNA and/or RNA) or proteins that are identical or similar across two or more species of organisms. These types of conserved sequences are also sometimes called orthologous sequences. Some conserved/orthologous sequences can be particularly highly conserved. A highly conserved sequence is one that has remained relatively unchanged relatively far back up the phylogenetic tree, and hence relatively far back in geological time.

This can lead to errors in the detection of a gene sequence that is conserved between multiple organisms that are included in reference libraries against which the results of a given sample are compared (which are sometimes referred to herein as reference organisms). For example, if a gene sequence is conserved between two reference organisms (e.g., Reference Organism A and Reference Organism B), the detection of said conserved gene sequence in a sample can result in a conclusion that Reference Organism A is present in the sample even though only Reference Organism B is actually present, or vice-versa.

Another, related source of potential false positives is symplesiomorphies, in which certain genetic material was present in a common ancestor, and is now a highly conserved gene sequence that is widely shared by many species. As a result, this highly conserved gene sequence can be present in numerous reference organisms. Such a highly conserved gene sequence can be misattributed to an organism that is not present in the sample, unless it is otherwise accounted for.

Another potential source of false positives is convergence and/or homoplasy, in which different organisms have portions of genetic sequences that match (and thus are similar to conserved gene sequences), even though the organisms are not closely related and the genetic sequence was not present in their common ancestor.

These sources of error can lead to results that indicate the presence of many organisms that are not present in a sample and/or are unlikely to be present in the sample.

Additionally, certain attempts accounting for these sources of error can themselves lead to other types of error (e.g., false negatives), such as results being reported that fail to indicate the presence of a certain reference organism(s) that are present in a sample and/or are likely to be present in the sample. One potential source of this type of error is an attempt to account for some conserved gene sequences by removing certain conserved gene sequences from the libraries that contain the gene sequence information for reference organisms, against which the results of a given sample are compared. Although removing certain conserved gene sequence(s) from said libraries can prevent said conserved gene sequence(s) from being misattributed to an organism that is not present in the sample (and thereby potentially prevent a false positive result), such a removal can also cause a false negative result. For example, a fragment of a gene sequence that is actually present in a sample and that actually belongs to a reference organism can go unidentified, because the conserved gene sequence that was removed from the library represents some or all of the fragment detected. Thus, because the reference library was intentionally depleted, a fragment gene sequence that actually belongs to a reference organism can go unidentified, even though the fragment sequence is detected in the sample and is generally known to be present in the reference organism. In some clinical situations, a false negative result is more problematic than a false positive result.

Moreover, because different organisms are diagnostically relevant at different concentrations, while various sources of error can lead to many false positive readings, in some situations low level results can be clinically/diagnostically relevant (e.g. signaling a True Positive). As such, the detection of fragments that only contain a conserved gene sequence cannot be ignored. For similar reasons, the detection of fragments in which a conserved gene sequence is a major component, or even the only identifiable component, cannot be ignored either.

The terms Limit of Blank (LoB), Limit of Detection (LoD), and Limit of Quantitation (LoQ) are used herein to describe certain points relating to smallest concentration of a measurand that can be reliably measured by an analytical procedure.

The term Limit of Blank (LoB) can be the highest apparent analyte concentration expected to be found when replicates of a blank sample containing no analyte are tested. LoB can be defined as the average signal of a given target concentration, recovered in 95% of replicates. This can be a baseline threshold for detection.

The term Limit of Detection (LoD) can be the lowest analyte concentration likely to be reliably distinguished from the LoB and at which detection is feasible. LoD is determined by utilizing both the measured LoB and test replicates of a sample known to contain a low concentration of analyte. LoD can often be defined as the average signal of target in Blanks/Target-negative Matrix+2 Standard Deviations. LoD can also be considered as representing the level of the ambient noise of a system for a given target. When measuring the concentration of an analyte, if the signal produced by the presence of the analyte is less than the analytical noise produced by the system being used to detect the presence of the analyte it is difficult to determine whether the resulting signal is a true positive. If the analyte concentration is relatively low (e.g., below the LoD), the analyte signal cannot be reliably distinguished from analytical noise. For this reason, a limit can be set for the detection of the analyte (LoD), which is higher than the signals that fall in the analytical noise zone. This can increase the likelihood a signal is indeed due the analyte, and not due the analytical noise.

As used herein, the term Limit of Quantitation (LoQ) is the lowest concentration at which a given analyte can not only be reliably detected but at which certain predefined goals for bias and imprecision can also be met. In certain situations, LoQ can be equivalent to LoD. However, in other situations, LoQ can be much higher than LoD. LoQ can be defined as the lowest average signal within a predefined level variance, as measured by percent coefficient of variation (% CV).

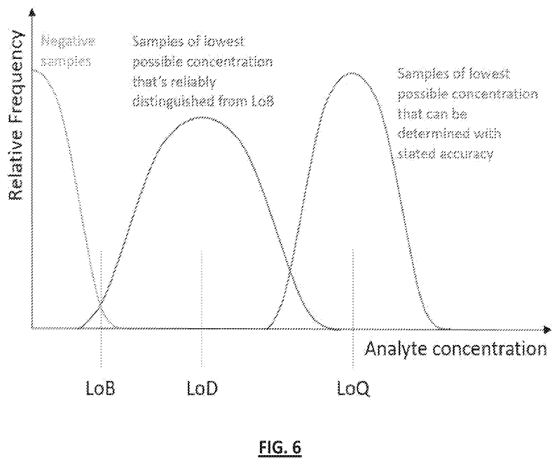

As described below, FIG. 6 shows a graphical representation of the relationship between LoB, LoD, and LoQ, with respect to measurand concentration.

As used herein, the term/abbreviation “Tb” refers to the signal threshold delineating true organism signal (e.g., a value derived from a sample that actually contains a given reference organism) from noise (e.g. values for the same given reference organism that are derived from samples that do not actually contain said reference organism).

The term/abbreviation “True Negative” or “TN” can refer to a sample with no target organism, and for which a target organism is not detected above threshold the relevant threshold (typically LoD and/or LoQ).

The term/abbreviation “False Positive” or “FP” can refer to a sample with no target organism, but for which a target organism is detected above threshold the relevant threshold (typically LoD and/or LoQ).

### Systems and Processes

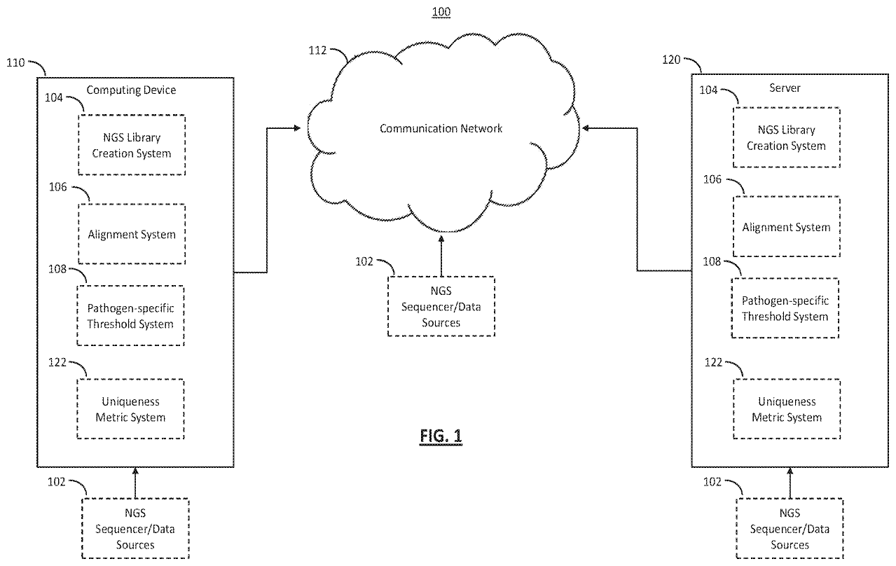

Referring now to the figures, FIG. 1 shows an example of a system for classifying genetic sequencing results based on pathogen-specific adaptive thresholds in accordance with some embodiments of the disclosed subject matter. As shown in FIG. 1, a computing device 110 can receive sequencing results indicating genetic information (e.g., DNA, RNA, etc.) that is present in a sample (e.g., a clinical sample, a negative control sample, a positive control sample) from a data source 102 that generated and/or stores such data, and/or from an input device. In some embodiments, computing device 110 can execute at least a portion of a Next Generation Sequence (NGS) Library Creation System 104, an alignment system 106, and/or a pathogen-specific threshold system 108.

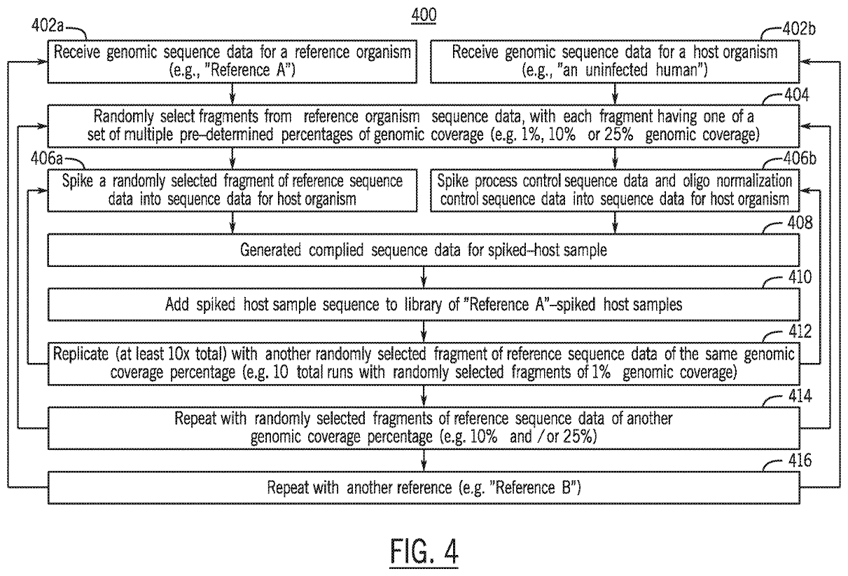

The NGS Library Creation System 104 can create and/or receive sequence data. In some embodiments, NGS Library Creation System 104 can generate new sequence data (e.g. “synthetic sequence data”) by modifying at least a portion of the sequence data received. In some embodiments, NGS Library Creation System 104 can generate synthetic sequence data by combining at least a portion of the sequence data associated with an organism with at least a portion of the sequence data associated with another organism. Moreover, NGS Library Creation System 104 can output a portion of the initially received sequence data, the synthetic sequence data, and/or a combination thereof in the form of an expanded library. For example, NGS Library Creation System 104 can execute one or more portions or versions of the process 400 described below in connection with FIG. 4.

In some embodiments, alignment system 106 can identify a correspondence between a read generated by a next generation sequencing device and a particular reference sequence (e.g., associated with a first pathogen, associated with a second pathogen, associated with both the first pathogen and the second pathogen, or associated with a likely source of contamination, etc.). In some embodiments, alignment system 106 can use any suitable alignment technique or combination of techniques, such as linear alignment techniques, and graph-based alignment techniques (e.g., as described in U.S. Patent Application Publication No. 2020/0090786, which is hereby incorporated by reference herein in its entirety).

In some embodiments, pathogen-specific threshold system 108 can generate a model (e.g., based on one or more negative control samples and/or positive control samples) that can be used to classify results associated with a particular pathogen as being consistent with negative controls (e.g., as being below a threshold), or as being indicative of presence of the pathogen in the sample being analyzed.

Additionally or alternatively, in some embodiments, computing device 110 can communicate information about genetic information (e.g., genetic sequence results generated by a next generation sequencing device, aligned reads associated with a particular reference sequence) from data source 102 to a server 120 over a communication network 112 and/or server 120 can receive genetic information from data source 102 (e.g., directly and/or using communication network 112), which can execute at least a portion of NGS Library Creation System 104, alignment system 106, a pathogen-specific threshold system 108, and/or a uniqueness metric system 122. In such embodiments, server 120 can return analysis results to computing device 110 (and/or any other suitable computing device) indicative of levels of one or more pathogens detected in a sample and/or a likelihood that the pathogen is a true positive in the sample.

In some embodiments, computing device 110 and/or server 120 can be any suitable computing device or combination of devices, such as a desktop computer, a laptop computer, a smartphone, a tablet computer, a wearable computer, a server computer, a virtual machine being executed by a physical computing device, a specialty device (e.g., a next generation sequencing device), etc. As described below, in some embodiments, computing device 110 and/or server 120 can receive genetic data (e.g., corresponding to a positive control sample, a negative control sample, or a clinical sample) from one or more data sources (e.g., data source 102), can create a sequence library (e.g., using NGS Library Creation System 104), can associate portions of the genetic data with one or more reference genomes (e.g., using alignment system 106), and/or can generate a model that that can be used to classify results associated with a particular pathogen and/or use the model to classify results associated with a particular pathogen using pathogen-specific threshold system 108. Additionally or alternatively, in some embodiments, computing device 110 and/or server 120 can receive genetic data (e.g., corresponding to a clinical sample, a positive control sample, a negative control sample, etc.) from one or more data sources (e.g., data source 102), can associate portions of the genetic data with one or more particular portions of one or more reference genomes (e.g., using alignment system 106), and can generate uniqueness metrics associated with pathogens and/or organisms associated with the particular portions of the one or more reference genomes based on reads that uniquely align to particular taxa represented I the one or more reference genomes.

In some embodiments, data source 102 can be any suitable source or sources of genetic data. For example, data source 102 can be a next generation sequencing device or devices that generate a large number of reads from a sample. As another example, data source 102 can be a data store configured to store genetic data, which can be aligned genetic data or unaligned reads.

In some embodiments, data source 102 can be local to computing device 110. For example, data source 102 can be incorporated with computing device 110. As another example, data source 102 can be connected to computing device 110 by one or more cables, a direct wireless link, etc. Additionally or alternatively, in some embodiments, data source 102 can be located locally and/or remotely from computing device 110, and provide data to computing device 110 (and/or server 120) via a communication network (e.g., communication network 112).

In some embodiments, communication network 112 can be any suitable communication network or combination of communication networks. For example, communication network 112 can include a Wi-Fi network (which can include one or more wireless routers, one or more switches, etc.), a peer-to-peer network (e.g., a Bluetooth network), a cellular network (e.g., a 3G network, a 4G network, a 5G network, etc., complying with any suitable standard, such as CDMA, GSM, LTE, LTE Advanced, WiMAX, 5G NR, etc.), a wired network, etc. In some embodiments, communication network 112 can be a local area network, a wide area network, a public network (e.g., the Internet), a private or semi-private network (e.g., a corporate or university intranet), any other suitable type of network, or any suitable combination of networks. Communications links shown in FIG. 1 can each be any suitable communications link or combination of communications links, such as wired links, fiber optic links, Wi-Fi links, Bluetooth links, cellular links, etc.

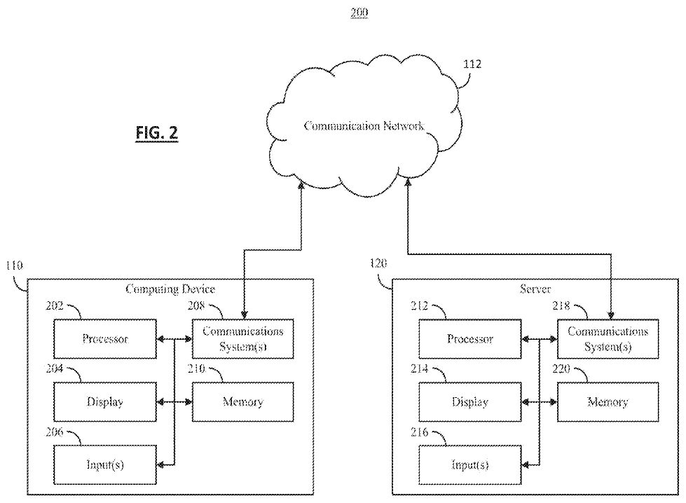

FIG. 2 shows an example 200 of hardware that can be used to implement computing device 110 and/or server 120, in accordance with some embodiments of the disclosed subject matter. As shown in FIG. 2, in some embodiments, computing device 110 can include a processor 202, a display 204, one or more inputs 206, one or more communication systems 208, and/or memory 210. In some embodiments, processor 202 can be any suitable hardware processor or combination of processors, such as a central processing unit (CPU), a graphics processing unit (GPU), a microcontroller (MCU), an application specification integrated circuit (ASIC), a field programmable gate array (FPGA), etc. In some embodiments, display 204 can include any suitable display devices, such as a computer monitor, a touchscreen, a television, etc. In some embodiments, inputs 206 can include any suitable input devices and/or sensors that can be used to receive user input, such as a keyboard, a mouse, a touchscreen, a microphone, etc.

In some embodiments, communications systems 208 can include any suitable hardware, firmware, and/or software for communicating information over communication network 112 and/or any other suitable communication networks. For example, communications systems 208 can include one or more transceivers, one or more communication chips and/or chip sets, etc. In a more particular example, communications systems 208 can include hardware, firmware and/or software that can be used to establish a Wi-Fi connection, a Bluetooth connection, a cellular connection, an Ethernet connection, etc.

In some embodiments, memory 210 can include any suitable storage device or devices that can be used to store instructions, values, etc., that can be used, for example, by processor 202 to present content using display 204, to communicate with server 120 via communications system(s) 208, etc. Memory 210 can include any suitable volatile memory, non-volatile memory, storage, or any suitable combination thereof. For example, memory 210 can include RAM, ROM, EEPROM, one or more flash drives, one or more hard disks, one or more solid state drives, one or more optical drives, etc. In some embodiments, memory 210 can have encoded thereon a computer program for controlling operation of computing device 110. In such embodiments, processor 202 can execute at least a portion of the computer program to present content (e.g., user interfaces, graphics, tables, reports, etc.), receive genetic data from data source 102, receive information (e.g., content, genetic information, etc.) from server 120, transmit information to server 120, etc.

In some embodiments, server 120 can include a processor 212, a display 214, one or more inputs 216, one or more communications systems 218, and/or memory 220. In some embodiments, processor 212 can be any suitable hardware processor or combination of processors, such as a CPU, a GPU, an MCU, an ASIC, an FPGA, etc. In some embodiments, display 214 can include any suitable display devices, such as a computer monitor, a touchscreen, a television, etc. In some embodiments, inputs 216 can include any suitable input devices and/or sensors that can be used to receive user input, such as a keyboard, a mouse, a touchscreen, a microphone, etc.

In some embodiments, communications systems 218 can include any suitable hardware, firmware, and/or software for communicating information over communication network 112 and/or any other suitable communication networks. For example, communications systems 218 can include one or more transceivers, one or more communication chips and/or chip sets, etc. In a more particular example, communications systems 218 can include hardware, firmware and/or software that can be used to establish a Wi-Fi connection, a Bluetooth connection, a cellular connection, an Ethernet connection, etc.

In some embodiments, memory 220 can include any suitable storage device or devices that can be used to store instructions, values, etc., that can be used, for example, by processor 212 to present content using display 214, to communicate with one or more computing devices 110, etc. Memory 220 can include any suitable volatile memory, non-volatile memory, storage, or any suitable combination thereof. For example, memory 220 can include RAM, ROM, EEPROM, one or more flash drives, one or more hard disks, one or more solid state drives, one or more optical drives, etc. In some embodiments, memory 220 can have encoded thereon a server program for controlling operation of server 120. In such embodiments, processor 212 can execute at least a portion of the server program to transmit information and/or content (e.g., a user interface, graphs, tables, reports, etc.) to one or more computing devices 110, receive genetic data, information, and/or content from one or more computing devices 110, receive instructions from one or more devices (e.g., a personal computer, a laptop computer, a tablet computer, a smartphone, etc.), etc.

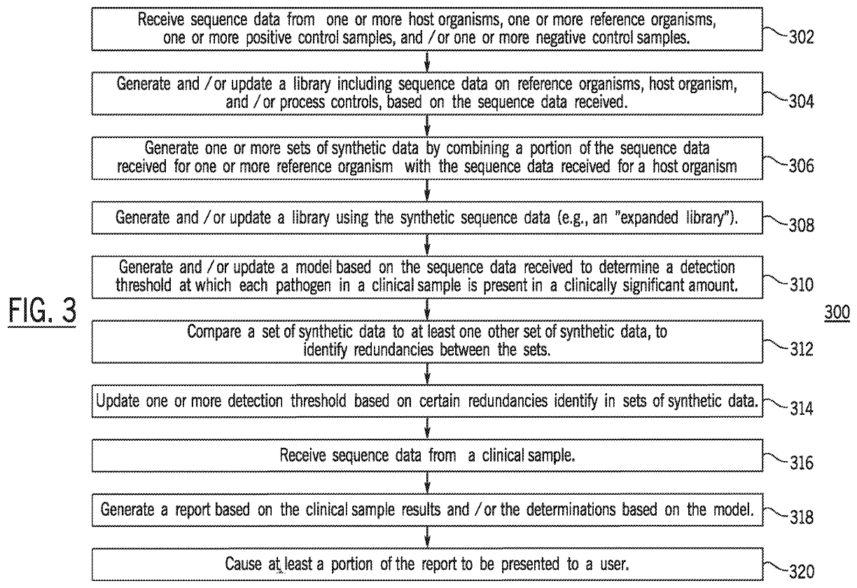

FIG. 3 shows an example 300 of a process for determining and/or optimizing sequencing results for pathogens having cross-reactivity capable of accounting for shared sequence information, in accordance with some embodiments of the disclosed subject matter.

At 302, process 300 can receive experimentally/clinically generated sequence data, such as gene sequence data, protein sequence data, or other similar data. The sequence data received can be representative of a sample from a host organism, a sample from a reference organism, or a sample of certain process control sequences. Process 300 can receive genetic data (e.g., genetic sequencing results) corresponding to one or more host organisms, one or more reference organisms, one or more process controls, one or more positive control samples, and/or one or more negative control samples.

In embodiments where the sequence data is gene sequence data, the sequence data can represent the entire genomic sequence of a host organism, the entire genomic sequence of a reference organism, only a fragment of the genomic sequence of a host, only a partial fragment of the genomic sequence of a reference organism, and/or any combination thereof.

In some embodiments, the sequence data can represent at least a portion of the genome of a host organism and/or at least a portion of the genome of a reference organism. Additionally, the sequence data can represent certain process control sequences.

In some embodiments, process 300 can receive sequence data that constitutes about 1% of the total genome of a pathogen. In some embodiments, process 300 can receive sequence data that constitutes about 3%, about 5%, about 10%, about 15%, about 20%, about 25%, about 33%, or about 50%, of the total genome of a pathogen. However, in a particular embodiment, the process 300 can receive sequence data representing the total genome of a reference organism (e.g. about 100%) and/or sequence data representing the all of the coding sections of the genome of a reference organism.

In some embodiments, the genetic data received at 302 can include any suitable information, and can be in any suitable format. For example, in some embodiments, the genetic data received at 302 can be formatted as results from a next generation sequencing device. In more particular example, the results can be formatted as a binary base call (BCL) file, which includes information received from the sequencer's sensors (e.g., regarding the luminescence that represent the biochemical signal of the reaction). In such an example, process 300 can include aligning the genetic data received at 302 (e.g., using alignment system 106). In such an example, the data can be converted into another format, such as a FASTQ format, that includes both a called base and a quality score for each position of a read. As another example, the genetic data received at 302 can be received as reads that include a called base and in some cases a quality score for each position of each read. In a more particular example, the results can be formatted a FASTQ file.

As another example, the genetic data received at 302 can be formatted as a raw count of reads associated with various pathogens (which can or can not be reference organisms) and/or other organisms, identifying information associated with a particular pathogen (and/or other organism), identifying information associated with a group of pathogens/other organisms (e.g., organized at any suitable taxonomic level, which is sometimes referred to herein as a taxon), and/or identifying information of reads associated with the pathogen and/or other organism (e.g., based on a reference sequence, based on a reference sequence with alternates, etc.). Note that the count of reads can be formatted in multiple ways. For example, the count of reads can be formatted as the total reads (which is sometimes referred to as alignments) that align to each pathogen or other organism, including repeats. As another example, the count of reads can be formatted as the count of reads that align uniquely to that pathogen or other organism, excluding reads that were observed multiple times. In some embodiments, the data received at 302 can be organized such that the data is grouped by taxon, and taxons of different taxonomic rank are represented in the data. For example, the data received at 302 can include values associated with particular pathogens (e.g., a taxon at a species or subspecies taxonomic level), and other values associated with a group of pathogens (e.g., a taxon at a genus, family, or order taxonomic level).

As yet another example, the genetic data received at 302 can be formatted as a statistical transform of raw counts. For example, the statistical transform can be based on the proportion of the total counts made up by counts associated with a particular pathogen (e.g., a ratio of reads for pathogen x to total reads, a normalized ratio of reads for pathogen x to total reads). As another example, the statistical transform can be based on uniqueness of the alignment (e.g., the value of the statistical transform can be inversely proportional to the number of other species the alignment maps to), the pathogen's informational complexity and how closely the read maps to a particular reference genome (e.g., the human genome for samples taken from a human). In such an example, reads that are more unique and/or that are more complex can be associated with higher values from the transform, while reads that map closely to the particular reference genome can be associated with a lower value.

In some embodiments, results associated with a control sample can be identified as being a positive control sample for one or more organisms, and/or a negative control sample for one or more organism. Note that a sample cannot be a positive control sample and a negative control sample for the same organism. However, it is possible for a sample to be a positive control sample for one pathogen/organism while also simultaneously being a negative control sample for a different pathogen/organism. For example, in some embodiments, metadata (e.g., a file name) associated with sequencing results of a sample can identify whether the sample is a positive control sample and/or a negative control sample, with respect to a specific organism. As another example, a location of sequencing results of a sample can be used to identify whether the sample is a positive control sample and/or a negative control sample. In a more particular example, a folder in a file system (e.g., of computing device 110) can be designated as being associated with negative control samples, while another folder in the file system can be designated as being associated with positive control samples, and yet another folder in the file system can be designated as being associated with positive clinical samples.

At 304, process 300 can generate and/or update a library based on the sequence data received at step 302. In some embodiments, the library can include one or more entries based on the sequence data of a reference organism (which can be, for example, a pathogen”), one or more entries based on the sequence data of a host organism(s), and/or one or more entries based on the sequence data of a process control. In some embodiments, the library can contain one or more entries based on a combination of a pathogen, a host organism, and/or a process control. For example, the library can contain one or more entries that represent a host organism that has been infected with a pathogen. Additionally, the library can contain one or more entries that represent a clinical sample taken from a host organism that has been infected with a pathogen, the sample further including sequence information from certain process controls. In some embodiments, the library can contain one or more entries based on a combination of a host organism with more than one pathogen. For example, the library can contain one or more entries that represent a host organism that is simultaneously infected with two or more given pathogens.

At 306, process 300 can generate and/or update sets of synthetic sequence data by combining a portion of the sequence data received for one or more reference organisms with the sequence data received for a host organism (e.g., using NGS Library Creation System 104). In some embodiments, NGS Library Creation System 104 can execute at least a portion of process 300 (e.g., including 304 and/or 306).

In some embodiments, process 300 can modify one or more portions of the experimentally generated sequence data received at 302 and/or can combine at least a portion of the experimentally generated sequence data with at least one other portion of certain experimentally generated sequence data. After modifying and/or combining the experimentally generated sequence data, process 300 can generate new sequence data that is different from the experimentally generated sequence data, which can be referred to as “synthetic sequence data.” In some embodiments, the sequence information received by process 300 at 302 (and/or at another process point) can itself be synthetic sequence data (e.g., can have been extrapolated from known/experimental information by a separate process, prior to being received by NGS Library Creation System 104) and process 300 can use said initial synthetic sequence data to generate additional synthetic sequence data.

In some embodiments, process 300 can generate one more sets of synthetic data by combining a portion of sequence data for a reference organism with the sequence data for a host organism. Said synthetic sequence data can represent a certain host organism that was comingled and/or infected with a certain reference organism (e.g., a host infected with a pathogen).

In some embodiments, one more sets of synthetic data are generated by combining less than the entire genome of a pathogen with the sequence data for a host organism. The pathogen sequence data can constitute any amount of the total genome of said pathogen. At 306, process 300 can combine host sequence data with pathogen sequence data that constitutes about 1% of the total genome of a pathogen. In some embodiments, the pathogen sequence data can constitute about 3%, about 5%, about 10%, about 15%, about 20%, about 25%, about 33%, or about 50%, of the total genome of a pathogen. However, it is also possible for process 300 to generate synthetic sequence data by combining host sequence data with pathogen sequence data that represents the total genome of a reference organism (e.g. about 100%) and/or the total of the coding regions of a reference organism.

In some embodiments, one or more sets of synthetic data are generated by individually combining sequence data for each pathogen in a set of multiple pathogens with the sequence data of a host organism. In some embodiments, the pathogen sequence data for each pathogen represents a specific amount of the pathogen's total genome. For example, for a set of three pathogens, sequence data that constitutes 1% of the genome of a first pathogen is combined with the sequence data of a host organism, and sequence data that constitutes 1% of the genome of a second pathogen is combined with the sequence data of a host organism, sequence data that constitutes 1% of the genome of a third pathogen is combined with the sequence data of a host organism. The same original sequence data for the host organism can be used in each case. In this manner, a library of synthetic sequence data for combinations of 1% pathogen sequence data and host sequence data can be generated. In some embodiments, libraries of synthetic sequence data for combinations of any given amount/percentage of pathogen sequence data and host sequence data can be generated.

In some embodiments, the synthetic sequence data can include sequence data that represents more than one reference organism as well as sequence data that represents a host organism (e.g., a host that is infected with two or more pathogens). As described above, such synthetic sequence data can include any suitable portion of the sequence data for each pathogen. Optionally, the synthetic sequence data can also include sequence data that represents certain process controls.

At 308, process 300 can generate and/or update a library using the synthetic sequence data (e.g., can generate and/or update an “expanded library”). In some embodiments, some or all of 308 can be executed using NGS Library Creation System 104. An expanded library can include any type of synthetic sequence data generated by process 300. In some embodiments, step 308 of process 300 can generate an expanded library including at least one example of synthetic data described herein. In some embodiments, step 308 of process 300 can generate an expanded library including more than one example of synthetic data described herein. In some embodiments, step 308 of process 300 can generate more than one expanded library.

In some embodiments, an expanded library can contain experimentally generated sequence data that was originally received by process 300 at 302 and synthetic sequence data that was generated by process 300 at 306. In some embodiments, an expanded library can contain only synthetic sequence data.

A specific example of an expanded library can contain a combination of (1) a library of experimental sequence data and/or synthetic sequence data for combinations of 1% pathogen sequence data and host sequence data, and (2) a library of experimental sequence data and/or synthetic sequence data for combinations of 10% pathogen sequence data and host sequence data, and (3) a library of experimental sequence data and/or synthetic sequence data for combinations of 25% pathogen sequence data and host sequence data, can be generated.

In some embodiments, the amount of synthetic sequence data generated by process 300 (and thus the amount of sequence data stored in expanded libraries) can be greater than the amount of experimental sequence data that is initially received by process 300, as measured by the number of total base pairs or the total number of reads in the synthetic sequence data as compared to the number of base pairs or the total number of reads in the experimentally generated sequence data. The amount of synthetic sequence data can be from about 2× to about 1000× greater than the amount of experimentally generated sequence data. In some embodiments, the amount of synthetic sequence data can be from about 5× to about 500× greater, or from about 10× to about 100×, or about 30× greater than the amount of experimentally generated sequence data.

At 310, process 300 can generate and/or update a model based on one or more results based on the sequence data received at 302 and/or based on the synthetic sequence data generated at 306 and 308. In some embodiments, some or all of 310 can be carried out using Pathogen-specific Threshold System 108. Moreover, in some embodiments, 310 can form a part of Pathogen-specific Threshold System 108.

In some embodiments, the model can be used to determine and/or update a threshold at which each reference organism (for example, each pathogen) in a clinical sample is to be considered clinically significant. In some embodiments, process 300 can generate any suitable type of model. For example, process 300 can generate one or more statistical models for various organisms (e.g., pathogens) based on one or more control samples. In such an example, the statistical model can be used to determine an explicit threshold for a particular pathogen (or other organism) at which a clinical sample can be considered clinically significant. In such an example, if a value in results from a clinical sample meets and/or exceeds the threshold for a particular pathogen, that pathogen can be considered positive (e.g., present) in the sample.

The model can be any model suitable for analyzing, extrapolating, graphing, and/or visualizing the relevant data. In some embodiments, process 300 can generate and/or update a probit model at 310. A probit model is a type of regression model where the dependent variable can take only two values (which is sometimes referred to as a binary response model), for example “infected” or “not infected.” A purpose of the model can be to estimate the probability that an observation with particular characteristics falls into a specified category. When viewed in the generalized linear model framework, the probit model employs a probit link function, which is most often estimated using the maximum likelihood procedure. Such an estimation is often referred to as a probit regression.

In many probit models, the LoB can be set to zero by definition and needs only to be verified by testing multiple negative samples and confirming that the 95th percentile is zero. Once probit analysis has been performed, using appropriate techniques, an evaluation of the initial probit model is performed, typically according to the chi-square goodness-of-fit test, at which the detection probability equals 95%, is determined and reported as the LoD. If the model fit was insufficient, additional data can (e.g., additional synthetic sequence data) and the probit analysis re-performed.

In some embodiments, process 300 can generate and/or update a linear regression model at 310.

In some embodiments, process 300 can generate a new model based on a portion of the synthetic sequence data generated at 306/308 and/or based on a combination of a portion of the synthetic sequence data generated at 306/308 and a portion of the experimental sequence data received at 302.

In some embodiments, one or more models can exist prior to the beginning of process 300, and process 300 can, at 310, update an existing model based on a portion of the synthetic sequence data generated at 306/308 and/or based on a combination of a portion of the on the synthetic sequence data generated at 306/308 and a portion of the experimental sequence data received at 302.

As another example, process 300 can generate and/or update a machine learning model for various organisms (e.g., pathogens) based on synthetic sequence data. In such an example, an output of the machine learning model can be indicative of whether a particular pathogen is present in the sample. In such an example, the machine learning model can not generate an explicit threshold in terms of a semantically meaningful value (e.g., raw read count, a statistical transform of raw read counts). However, a threshold can be applied to the output of the machine learning model (e.g., for each pathogen). In a more particular example, the output for each pathogen can be a value in a range [0,1] (e.g., where higher numbers indicate a higher likelihood of the value indicating the presence of the corresponding pathogen). A threshold can be selected for the output (e.g., at 0.5, 0.75, 0.9, etc.), where an output that is at or above the threshold indicates a positive result for that pathogen, and a value under the threshold indicates a negative result for that pathogen.

Note that in some embodiments, process 300 can generate a statistical model at 310 based on experimentally generated sequence data, synthetic sequence data, and/or a combination thereof. For example, a kernel density estimation-based model can be based on clinical sample (e.g., experimental) results.

Once an initial model exists (e.g., prior to process 300) and/or has been generated (e.g., at 310), at 312, process 300 can compare a set of synthetic data to at least one other set of synthetic data, to identify redundancies in sequence information between the sets.

In some embodiments, some or all of 312 can be carried out using Alignment System 106. Moreover, in some embodiments, 312 can form a part of Alignment System 106.

For example, some or all of the experimental sequence data and/or the synthetic sequence data can be processed to generate “Species×Species matrices” of replicate-averaged signal for each genome coverage with paired covariance matrices. In some embodiments, some or all of 312 can use alignment system 106 to generate a Species×Species matrix. The Species×Species matrix can be generated for a given target at a given concentration, across multiple replicates. In some embodiments, the ‘target’ can be a portion of the genome of an organism. In some embodiments, the target can be the entire genome of an organism. In some embodiments, the, the ‘target’ can be a portion of the genome of more than one organism. For example, the Species×Species matrix can be generated using any suitable number of replicates (e.g., 100 replicates per milliliter, or 500 replicates per milliliter, or 1,000 replicates per milliliter, or 5,000 replicates per milliliter, or 10,000 replicates per milliliter, or 25,000 replicates per milliliter, or 100,000 replicates per milliliter), including separate matrix entries for each of several different numbers of replicates for the same species, for a given target at a given concentration. In some embodiments, some or all of 312 can use alignment system 106 to generate a Species×Species matrix across 10,000 replicates for a given target at a given concentration. In some embodiments, some or all of 312 can use alignment system 106 to average the value of the signal for each of 10,000 replicates for a given target at a given concentration. For example, in a particular matrix each row can represent a specific organism (e.g. Organism A, Organism B, Organism C, etc.) at a specific concentration of replicates per milliliter (for example, at 10,000 replicates per milliliter), and each column can represent a particular species. For example, if row 1 represents Organism A and column 1 represents Species 1, the value at the intersection of row 1/column 1 represents amount of Species 1 biomarker/genome that this present in the sample of Organism A (i.e. the signal strength of Species 1 presented by Organism A).

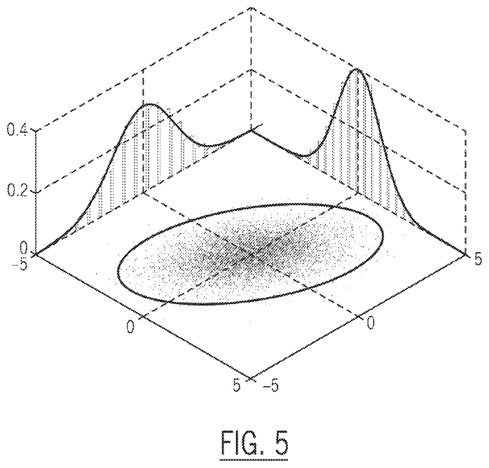

An example of such a “Species×Species matrix” of replicate-averaged signal is show in FIG. 5. The example in FIG. 5 shows a particular Species×Species matrix of replicate-averaged signal with paired covariance matrices in accordance with some embodiments of the disclosed subject matter. In the example shown in FIG. 5, inputs are shown along the Y-axis and categories for outputs are shown along the X-axis, while values for signal are shown along the Z-axis. In some embodiments, inputs shown along the Y-axis can be a particular organism (e.g. Organism A, Organism B, Organism C, etc.). Moreover, the inputs shown along the Y-axis can be based on experimentally generated sequence data, synthetic sequence data, and/or a combination thereof. For example, the inputs shown along the Y-axis can be experimentally generated sequence data. As another example, in other embodiments, the inputs shown along the Y-axis can be synthetic sequence data. In some embodiments, the outputs shown along the X-axis can be specific, known species of microorganism (e.g. Microorganism Species 1, Microorganism Species 2, Microorganism Species 3, etc.). The outputs shown along the X-axis can be based on experimentally generated sequence data, synthetic sequence data, and/or a combination thereof. In some embodiments, the outputs shown along the X-axis can be experimentally generated sequence data. In some embodiments, the outputs shown along the X-axis can be experimentally generated sequence data. In some embodiments, if the inputs shown along the Y-axis are experimentally generated sequence data, then the outputs shown along the X-axis are synthetic sequence data. In some embodiments, if the inputs shown along the Y-axis are synthetic sequence data, then the outputs shown along the X-axis are experimentally generated sequence data. The signal value shown along the Z-axis can be unitless and/or normalized. In some embodiments, the signal strength can represent the number of reads of a particular input (such as Organism A) corresponding to a particular output (such as Species 1). For example, any of the spiked-host sample entries contained in a Reference Spiked-Host Library (e.g., described below with respect to process 400 shown in FIG. 4), can be processed to generate said Species×Species matrices of replicate-averaged signal. In some embodiments, spiked-host sample entries including the same amount of sequence data for their respective reference organisms (e.g. a host organism spiked with a pathogen) are processed/compared to generate “Species×Species matrices” of replicate-averaged signal for each genome coverage with paired covariance matrices. For example, a spiked-host sample entry that contains 1% of sequence data for Reference Organism A can be processed with a spiked-host sample entry that contains 1% of sequence data for Reference Organism B, to generate a Species×Species matrix of replicate-averaged signal.

In some embodiments, these signal distributions can be represented as joint probability density functions (e.g., “p(A)=p(Species₁=value₁, Species₂=value₂, Species₃=value₃ . . . , Speciesₓ=valueₓ)”).

At 314, process 300 can generate and/or update one or more detection threshold(s) of a model. Additionally or alternatively, process 300 can compare the inputs and outputs of a Species×Species Matrix to one or more other Species×Species Matrices. Additionally or alternatively, process 300 can update of the one or more detection threshold(s) of a model, based on a comparison between a Species×Species Matrix to one or more other Species×Species Matrices. In some embodiments, the detection threshold(s) generated/updated are selected from the group including an LoB, an LoD, an LoQ, and combinations thereof. In some embodiments, the detection threshold generated/updated is an LoD and/or an LoQ, which can be equivalent. In some embodiments, the detection threshold generated/updated is an LoD. In some embodiments, the detection threshold generated/updated is an LoQ.

In some embodiments, some or all of 314 can be carried out using Pathogen-specific Threshold System 108. Moreover, in some embodiments, 314 can form a part of Pathogen-specific Threshold System 108

In some embodiments, one or more detection threshold(s) of a model can be generated and/or updated based on a statistical analysis of the covariance between one or more pair(s) of sequence information. In some embodiments, one or more inputs (i.e. a set of inputs) can be compared to one or more outputs (i.e. a set of outputs) using a statistical analysis of the covariance between signal strength of the set of inputs and the set of outputs, to determine which set of inputs most closely corresponds to the observed signal strength of the output(s).

In some embodiments, the sequence information can include synthetic sequence information. For example, in some embodiments, one or more detection threshold(s) of a model can be generated and/or updated based a statistical analysis of the covariance between one or more pair(s) of spiked-host sample entries from one or more Reference Spiked-Host Library (as described below with respect to process 400 shown in FIG. 4). In particular, one or more detection threshold(s) of a model can be generated and/or updated based a statistical analysis of ones or more Species×Species matrices of replicate-averaged signal that are themselves derived from one or more pair(s) of spiked-host sample entries (which themselves are each synthetic sequence data).

In some embodiments, the statistical analysis used to generate and/or update a detection threshold can be based on conditional probability. In some embodiments, the statistical analysis used to compare the inputs and outputs of one or more Species×Species matrices can be based on conditional probability. For example, the statistical analysis used can be based on a Bayesian statistical analysis. In some embodiments, the statistical analysis used can use Bayes theorem, which can be represented as:

$$P ( B \vert A ) = \frac{P ( A \vert B ) P ( B )}{P ( A )}$$

where P(B|A) is the probability that target (e.g. the reference organism or the pathogen) was detected given the estimated prior signal distribution. As described above, in some embodiments, joint probability density functions can be generated by and/or derived from one or more “Species×Species matrices” of replicate-averaged signal. For example, in some embodiments, the value p(A) can be set using “p(A)=p(Species₁=value₁, Species₂=value₂, Species₃=value₃ . . . , Speciesₓ=valueₓ).”

In some embodiments, the statistical analysis can be used to compare the signal distribution of a sample to one or more other signal distributions. For example, in some embodiments, the statistical analysis can use one or more joint probability density functions to compare the signal distribution of a sample to one or more other signal distributions. In a more particular example, the statistical analysis can use one or more joint probability density functions to compare the signal distribution of an input that is based on experimentally generated sequence data, to one or more other signal distributions. In such an example, the one or more other signal distributions can include synthetic sequence data.

In some embodiments, the statistical analysis can be used to identify a set of inputs that most closely corresponds to the observed outputs in the sample. For example, in some embodiments, the inputs can be synthetic sequence data and the outputs can be experimentally generated sequence data (such as a sample from a subject, processed in a lab). In a more particular example, the inputs can be the signal strength of one or more known microorganism species (such as the signal strength for an idealized in silico model for said known microorganism species) and the outputs can be the signal strength of one or more unknown microorganism species (such as one or more unknown microorganism species present in a ‘real’ sample, which was taken from a subject and processed in a laboratory). In such an example, the statistical analysis can be used to determine (e.g., using a Species×Species matrix), which set of idealized in silico inputs most closely corresponds to the signal strength(s) of the outputs observed from the ‘real’ sample.

In some embodiments, the statistical analysis used to compare the inputs and outputs and/or to generate and/or update a detection threshold can be a loss function (also known as a cost function). At a high level, in situations where multiple microorganisms produce or are represented by very similar Species×Species matrices (e.g., where multiple microorganisms have a high degree of homogeneity or are very homologous), the Species×Species matrices of said microorganisms can be compared to numerous different Species×Species matrices and one or more loss functions can be used to analyze the degree of correspondence between the Species×Species matrices. In some embodiments, a loss function can be used to optimize between the joint density of the unknown estimate and the prior estimate (e.g. the known estimate) to determine which distribution (and associated label) minimizes the distances between the two. In some embodiments, the loss function can be a classification loss function. In some embodiments, the loss function can be a regression loss function. In some embodiments, the loss function can be a Hinge Loss Function (also known as a Multi class SVM Loss Function), and/or Cross Entropy Loss Function. In some embodiments, the loss function can be a Mean Square Error Function, Mean Absolute Error Function, and/or a Mean Bias Error Function.

A Mean Square Error Function can be represented by the following equation:

$$\begin{matrix}Mean Squared Error & \\ MSE = \frac{{\sum }_{i = 1}^{n} {( {y}_{i} - {\hat{y}}_{i} )}^{2}}{n} & \end{matrix}$$

A Mean Absolute Error Function can be represented by the following equation:

$$\begin{matrix}Mean absolute error & \\ MAE = \frac{{\sum }_{i = 1}^{n} \vert {y}_{i} - {\hat{y}}_{i} \vert}{n} & \end{matrix}$$

A Mean Bias Error Function can be represented by the following equation:

$$\begin{matrix}SVM Loss or Hinge Loss & \\ SVM Loss = {\sum }_{j \neq {y}_{i}} max ( 0 , {s}_{j} - {s}_{{y}_{i}} + 1 ) & \end{matrix}$$

A Hinge Loss Function can be represented by the following equation:

$$SVM Loss = {\sum }_{j \neq {y}_{i}} max ( 0 , {s}_{j} - {S}_{{y}_{i}} + 1 )$$

$$SVM Loss or Hinge Loss$$

A Cross Entropy Loss Function can be represented by the following equation:

$$Cross Entropy Loss = - ( {y}_{i} log ( {\hat{y}}_{i} ) + ( 1 - {y}_{i} ) log ( l - {\hat{y}}_{i} ) )$$

$$Cross entropy loss$$

At 316, process 300 can receive genetic data associated with a clinical sample (e.g., from data source 102, from alignment system 106). In some embodiments, the genetic data can be formatted in any suitable format. For example, the genetic data received at 316 can be formatted in a format described above.

In some embodiments, the statistical analysis used to generate and/or update a detection threshold can be a combination including a Bayesian statistical analysis and one or more loss functions. In some embodiments, the statistical analysis used to compare the inputs and outputs of one or more Species×Species matrices can be a combination including a Bayesian statistical analysis and one or more loss functions.

In some embodiments, the statistical analysis used to generate and/or update a detection threshold can be implemented via a machine learning model, such as a neural network model. In some embodiments, the statistical analysis used to compare the inputs and outputs of one or more Species×Species matrices can be implemented via a machine learning model, such as a neural network model. In some embodiments, any suitable machine learning model can be used to implement the statistical analysis. In some embodiments, a machine learning model used to implement the statistical analysis can be an unsupervised machine learning model. In some embodiments, a machine learning model used to implement the statistical analysis can be a supervised machine learning model.

At 318, process 300 can use a model involved in any of 308, 310, 312, and/or 314 to determine, for each pathogen represented in the clinical sample results (and/or each pathogen of interest), whether the result is likely clinically significant. For example, if the model is used to generate an explicit threshold for various pathogens, process 300 can determine whether the clinical results for a particular pathogen meet or exceed the explicit threshold. As another example, if the model is a machine learning model, the clinical results can be provided as input to the machine learning model (e.g., a neural network) and an output(s) of the machine learning model can be used to determine a likelihood that each pathogen is clinically significant. In a more particular example, a value associated with a pathogen or group of pathogens can be provided as input to an input node associated with the pathogen or group of pathogens. An output from a corresponding output node can be a prediction of whether the value associated with the pathogen or group of pathogens represents a signal (e.g., the pathogen or one or more pathogens in the group of pathogens is present in the sample) or noise (e.g., the pathogen or one or more pathogens in the group of pathogens is not present in the sample). The output of the machine learning model can be formatted as a value in a range of zero to one, with values closer to zero indicating a greater likelihood that the pathogen is not present in the sample, and values closer to one indicating a greater likelihood that the pathogen is present in the sample. As yet another example, if the model is a statistical model based on the clinical sample, process 300 can determine whether a particular pathogen is likely to be clinically significant based on the model (e.g., based on a kernel density estimate, etc.).

Also at 318, process 300 can generate a report based on the clinical sample results, the one or more determinations made based on the model, and/or the one or more control sample results. In some embodiments, the report can include any suitable content, information, and/or data. For example, the report can include a list of pathogens (if any) that are likely to be clinically significant. As another example, the report can include information indicating confidence in the classification of any positive results. As yet another example, the report can include graphics (e.g., one or more heatmaps, one or more boxplots, etc.) indicative of the results generated for the clinical sample and/or one or more control samples. As still another example, the report can include a list of pathogens that are unlikely to be clinically significant and/or a list of pathogens for which clinical significance is unclear.

At 320, process 300 can cause at least a portion of the report to be presented to a user. For example, in some embodiments, process 300 can cause a computing device (e.g., computing device 110) to present at least a portion of the report to a user. In some embodiments, process 300 can cause the report or a portion thereof to be presented in response to a request. As another example, process 300 can cause the report to be sent to an inbox (e.g., an email account, a healthcare messaging platform, and/or a cellular text messaging service) or other storage location from which the report can be retrieved (e.g., for analysis by a user).

FIG. 4 shows an example 400 of a process for generating synthetic sequence data sets and/or expanded libraries in accordance with some embodiments of the disclosed subject matter. At 402a, process 400 can receive genomic sequence data for a given reference organism (e.g. “Reference Organism A”). At 402b, process 400 can receive genomic sequence data for a host organism (e.g. “an uninfected human”). In some embodiments, the sequence data for the reference organism and/or the host organism can represent the entire genome of said organism. Alternatively, in some embodiments, the sequence data for the reference organism and/or the host organism can represent only part of the genome of said organism, such as only the coding regions of the genome. In some embodiments, 402a and 402b can be executed in parallel. Alternatively, in some embodiments, 402a and 402b can be executed serially (e.g., 402a can be executed before or after 402b).

At 404, process 400 randomly selects numerous fragments from the sequence data of the reference organism, with each fragment having a length that is one of a set of multiple pre-determined percentages of genomic coverage. For example, each fragment of reference organism sequence data has a length that represents a certain percentage (e.g., 1%, 10% 25%, etc.) of the total genomic sequence data for said reference organism (e.g. Reference Organism A).

At 406a, process 400 can spike a randomly selected fragment of reference organism sequence data into the sequence data for the host organism. The fragment of reference organism sequence data that is spiked into the host sequence data can have any of the pre-determined lengths (e.g. a fragment representing 1% of the sequence data of the reference organism can be spiked into the host sequence data, or alternatively, a fragment representing 10% of the sequence data of the reference organism can be spiked into the host sequence data, or alternatively, a fragment representing 25% of the sequence data of the reference organism can be spiked into the host sequence data). At 406b, process 400 can spike process control sequence data and/or oligo normalization control sequence data into sequence data for host organism. In some embodiments, 406a and 406b can occur simultaneously. Alternatively, in some embodiments, 406a can occur either before or after 406b.

At 408, process 400 can generate a compiled version of the sequence data for the spiked-host sample. This compiled version of said sequence data can be referred to simply as the spiked-host sample or the spiked-host sequence data. In some embodiments, the compiled version of the sequence data for spiked-host sample can contain sequence data from a reference organism that represents 1% or 10% or 25% of the total sequence data for said reference organism. In some embodiments, the compiled version of the sequence data for the spiked-host sample can contain sequence data from a process control and/or an oligo normalization control. In some embodiments, the compiled version of the sequence data for the spiked-host sample can contain both sequence data from a reference organism and sequence data from a process control and/or an oligo normalization control. The spiked-host sequence data is an example of synthetic sequence data.

At 410, process 400 adds sequence data for the spiked-host sample to a library (or sub-library) that contains or will contain a plurality of entries, where each entry represents sequence data for a spiked-host sample, where the reference organism that is spike into the host organism is “Reference A”. For example, such a library or sub-library can be referred to as “Reference A Spiked-Host Library.” In some embodiments, some entries in the Reference A Spiked-Host Library can contain sequence data for Reference Organism A that represents a different amount of the total sequence data for said Reference Organism A than that of a different entry.

At 412, process 400 repeats and/or replicates at least a portion of process 400 that was previously performed. In some embodiments, at 412, process 400 randomly selects another fragment of sequence data for the same reference organism (e.g. Reference Organism A). In some embodiments, at 412, process 400 repeats at least a portion of the process 400 beginning at 404a/404b. In some embodiments, at 412, process 400 randomly selects another fragment of sequence data for the same reference organism (e.g. Reference Organism A) and spikes said fragment into the sequence data for the same host organism. In some embodiments, at 412, process 400 can repeat/replicate at least a portion of the process 400 beginning at 406a/4046.

For example, in some embodiments, Reference A Spiked-Host Library can include a fourth spiked-host sample entry that has sequence data that represents 1% of the total sequence data for said Reference Organism A, and a fifth spiked-host sample entry that has sequence data that also represents 1% of the total sequence data for said Reference Organism A. In some embodiments, the reference organism sequence data of the fourth spiked-host sample entry can represent a different 1% of the total sequence data for said Reference Organism A than the reference organism sequence data of the fifth spiked-host sample entry.

In some embodiments, at 412, process 400 can be replicated more than once, with each replication returning to 404a/404b and/or 406a/406b, such that each replication spikes a different randomly selected fragment of sequence data for Reference Organism A into the sequence data for the host organism. In some embodiments, at least 10 replication can be performed, with each replication using a unique fragment of sequence data that represents a different 1% of Reference Organism.

Process 400 can add each replication to a Reference Spiked-Host Library at 410. For example, in some embodiments, Reference A Spiked-Host Library can include a ten spiked-host sample entries that each has sequence data that represents a unique 1% of the total sequence data for said Reference Organism A.

At 414, a portion of process 400 can be further repeated/replicated, using sequence data for the same reference organism (e.g. Reference Organism A) that represents a different amount of the total sequence data for said reference organism (e.g. 10%, 20%, 25%, 30%). In some embodiments, at 414, the replications described above with respect to 412 can themselves be repeated using randomly selected fragments of reference organism sequence data of a different length. For example, in some embodiments, at least 10 replication can be performed using sequence data that represents 10% of the total sequence data of the reference organism.

Process 400 can also add each replication performed at 414 to a Reference Spiked-Host Library at 410. For example, in some embodiments, Reference A Spiked-Host Library can also include ten spiked-host sample entries that each has sequence data that represents a unique 10% of the total sequence data for said Reference Organism A (in addition to the previously added ten spiked-host sample entries that each has sequence data that represents a unique 1% of the total sequence data for said Reference Organism A).

In some embodiments, process 400 can be repeated/replicated using multiple different lengths of sequence data. For example, at 414, the replications discussed above with respect to 412 can be repeated using randomly selected fragments of reference organism that represent 10% of the total sequence data of the reference organism and further also using randomly selected fragments of reference organism that represent 25% of the total sequence data of the reference organism.

For example, in some embodiments, following step 414, Reference A Spiked-Host Library can include one or more spiked-host sample entries that each have sequence data that represents 1% of the total sequence data for said Reference Organism A, and one or more spiked-host sample entries that each have sequence data that represents 10% of the total sequence data for said Reference Organism A, and one or more spiked-host sample entry that each have sequence data that represents 25% of the total sequence data for said Reference Organism A.

Process 400 can update a Reference Spiked-Host Library such that said library contains any suitable or necessary number of spiked-host sample entries. In some embodiments, a Reference Spiked-Host Library can contain a combination of multiple spiked-host sample entries, with each having sequence data that represents a first, a second, and/or a third percentage of the total sequence data for a Reference Organism (e.g. multiple entries with each having 1% of Reference Organism A). For example, a Reference A Spiked-Host Library can have 10 entries that each include a unique fragment of sequence data representing 1% of Reference Organism A, and 10 entries that each include a unique fragment of sequence data representing 10% of Reference Organism A, and 10 entries that each include a unique fragment of sequence data representing 25% of Reference Organism A. Therefore, this example Reference A Spiked-Host Library would have 30 spiked-host sample entries. It is possible to form this example Reference A Spiked-Host Library from a single sample of reference organism A and a single sample of the host organism, using process 400. In some embodiments, the initial sample of reference organism A and the initial simple of the host organism can be generated experimentally or clinically (e.g. are experimental/clinical sequence data. In some embodiments, each of the spiked-host sample entries can be generated synthetically, for example by process 400 (e.g. are synthetic sequence data). Therefore, in this example, Reference A Spiked-Host Library would represent a 30× increase in total sequence data as a result of synthetic sequence data generated by process 400.

At 416, some or all of process 400 can be repeated using a different reference organism (e.g. Reference Organism B). For example, another library or sub-library can be generated for said Reference Organism B (e.g. Reference B Spiked-Host Library). In some embodiments, some or all of process 400 can be further repeated using further reference organisms (e.g. Reference Organism C, D, E, etc.).

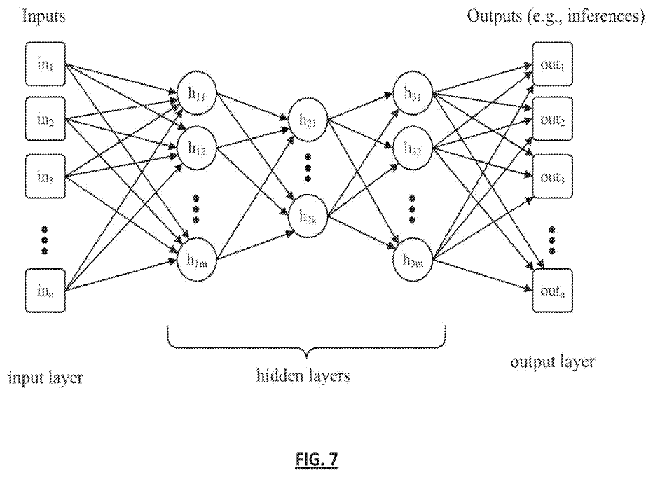

FIG. 7 shows an example of a topology of an autoencoder that can be trained to predict pathogen-specific adaptive thresholds for pathogens with cross reactivity using mechanisms described herein in accordance with some embodiments of the disclosed subject matter. In general, an autoencoder can include an input layer, one or more hidden layers, and an output layer (generally having the same number of nodes as the input layer). Each layer of an autoencoder can be fully connected. For example, as shown in FIG. 7, each node in the input layer is connected to each node in the first hidden layer, and each node in the first hidden layer is connected to each node in the next hidden layer, etc. In some embodiments, an autoencoder trained using mechanisms described herein can include an input node associated with each organism (e.g., pathogen) or group of organisms grouped at any suitable taxonomic level (or levels). For example, each input node can correspond to a particular species or sub-species (or any other suitable taxonomic grouping at or below genus), or a variant within a species or subspecies (e.g., a strain). As another example, each input node can correspond to a particular genus. As yet another example, input nodes can correspond to different taxonomical groupings. In a more particular example, some input nodes can correspond to a species, other input nodes can correspond to a sub-species, and yet other nodes can correspond to a genus.

In some embodiments, the autoencoder can be trained with any suitable number of input nodes corresponding to any suitable organisms of interest. For example, the input layer can include thousands of input nodes. In a more particular example, the number of input nodes n represented in FIG. 7 can be over 1,000 input nodes, over 2,000 input nodes, over 3,000 input nodes, over 4,000 input nodes, over 5,000 input nodes, etc., with each node representing a particular pathogen or group of pathogens.

As another example, the input layer can include fewer than 1,000 input nodes (e.g., in a range including 100 and 900 nodes, in a range including 200 and 800 nodes, in a range including 300 and 700 nodes, in a range including 400 and 600 nodes, in a range including 450 and 550 nodes). In a more particular example, the input layer can include 93540 nodes.

In some embodiments, the autoencoder can be configured to include an output node corresponding to each input node. For example, each output node can correspond to a particular organism or group of organisms, and an output can correspond to a prediction of whether that organism is present in a sample.

The relatively simple topology shown in FIG. 7 includes an input layer, three symmetric hidden layers (having m, k, and m nodes, respectively), and an output layer. For example, the input layer can include n input nodes that are configured to receive a floating point input (e.g., representing a raw read count associated with a particular pathogen or group of pathogens, or a statistical transform of such a raw read count), a first hidden layer can include m nodes that are each connected to an output of every input node, a second hidden layer (which is sometimes referred to herein as a coding layer) can include k nodes that are connected to an output of every node in the first hidden layer, were k is less than m and less than n. A third hidden layer can include m nodes that are each connected to an output of every node in the coding layer (note that hidden layers that precede the coding layer are sometimes referred to as encoding layers, and hidden layers that follow the coding layer are sometimes referred to as decoding layers). An output layer can include n output nodes that are each connected to every node in the third hidden layer, and each can be configured to output a value that predicts whether the value provided at the corresponding input node exceeds a threshold. As described below, an encoder can be configured asymmetrically (e.g., with more hidden layers on one side of the coding layer than the other).

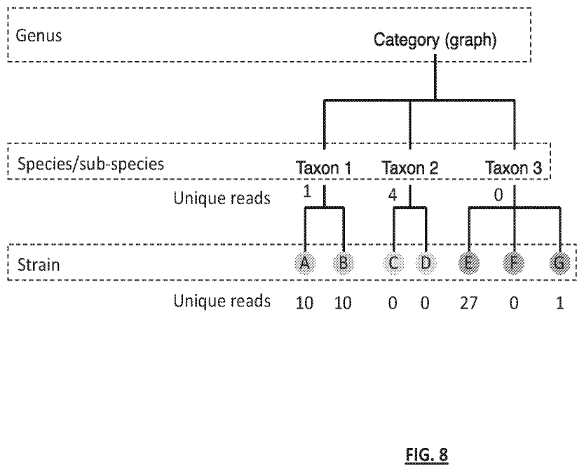

FIGS. 8-12B are related to mechanisms for classifying genetic sequencing based on the number of reads in the sequencing results that uniquely align to a particular taxa in accordance with some embodiments of the disclosed subject matter.

In accordance with some embodiments of the disclosed subject matter, mechanisms described herein can be used to generate a uniqueness metric based on genetic sequence results that can be used as an indication of whether a particular result (e.g., indicating that a particular pathogen and/or organism is present in a clinical sample) is a true positive or a false positive.

As described above, a sample (e.g., blood, sputum, fecal matter, etc.) can be sequenced to attempt to identify organisms present in the sample. Next generation sequencing techniques can be used to identify reads present in the sample relatively inexpensively and relatively quickly (e.g., on the order of dozens to thousands of base pairs in length). The reads can then be aligned to reference sequences associated with for various organisms to attempt to identify which organism a particular read originated from.

In some embodiments, different portions of a reference sequence (e.g., a graph reference, for example, as described in U.S. Patent Application Publication No. 2020/0090786, which has been incorporated by reference herein) can be associated with different taxa. For example, one or more portions of a reference sequence can be associated with a particular species or group of species, and one or more other portions (e.g., alternate paths) can be associated with particular sub-species and/or strains within a species.

In general, when attempting to identify whether a pathogen and/or other organism (e.g., a bacteria, a virus, a fungus, etc.) is present in a sample from a subject (e.g., a human subject), it can be assumed that there is zero, one, or an otherwise small number of pathogens present in the sample and that there are not multiple closely-related pathogens (e.g., due to co-infection by multiple relatively closely related strains) present in the sample (which can sometimes be referred to as the needle-in-the-haystack assumption). Under this assumption, it can be assumed that reads that, because there is likely to be at most a small number (e.g., one, two, three, etc.) pathogens present in the sample, reads that are present in the sample must be associated with a pathogen(s) that is present. If the sample is expected to have multiple relatively closely related strains and/or a relatively large number of pathogens (e.g., a sample taken from wastewater), the needle-in-the-haystack assumption may be relatively unlikely to hold, and classification based on a uniqueness metric may not be as useful.

As described above, various sources of error can cause false positive results to be included in the aligned reads (e.g., conserved sequences, convergence and/or homoplasy, contamination, etc.). In some embodiments, using a uniqueness metric described herein can lead to more precision in detection of pathogens in a sample (e.g., especially a sample that can be expected to meet the needle-in-the-haystack assumption).

FIG. 8 shows an example representation of a graph associated with a particular type of organism(s) with multiple taxonomic levels, and an indication of a number of reads from a sample that uniquely map to each taxa within a taxonomic level in accordance with some embodiments of the disclosed subject matter. In FIG. 8, a portion of a graph reference is shown in which multiple different taxonomical levels are represented (genus, species/sub-species, and strain in the example of FIG. 8). In the example of FIG. 8, sequence data from a clinical sample included reads that uniquely mapped to different strains (in the strain taxonomic level) represented in the graph, and strains that uniquely mapped to higher level taxonomic groups. A read that uniquely maps to a strain or other taxon can be a read that matches a portion of a reference associated with a particular member of a taxonomic level (e.g., a particular strain), and does not match any other members of that taxonomic level. In the particular example of FIG. 8, there are reads that are unique at the species/sub-species level (e.g., 1 read that is unique to taxon 1, and 4 reads that are unique to taxon 2), which can be reads that map to multiple strains encompassed by the species/sub-species (e.g., a read that maps to strain A and strain B can be unique to Taxon 1).

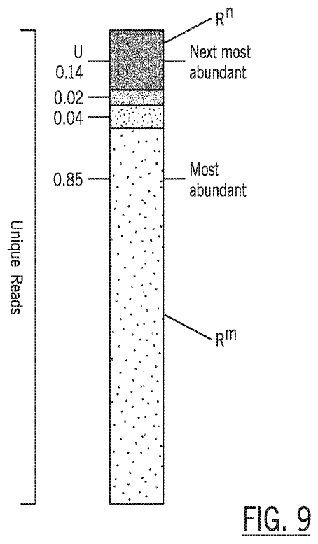

FIG. 9 shows an example representation of proportions of a unique reads that map to various taxa within a taxonomic level in accordance with some embodiments of the disclosed subject matter. In FIG. 9, there are four members of the taxonomic level (e.g., strains, species, etc.) that are associated with unique reads. The proportions at which unique reads are associated with the different members of the taxonomic level can be indicative of whether a particular member of the taxonomic level is actually present in the sample (e.g., indicative of whether the reads that map to that particular member of the taxonomic level represent a true positive or a false positive).

In some embodiments, mechanisms described herein can calculate a uniqueness metric that is based, in part, on the homogeneity of the resulting unique reads. For example, a homogeneity metric associated with the results at a particular taxonomic level (e.g., the strain level, the species level, the sub-species level, etc.) can be calculated based on the taxa (e.g., strain, species, etc.) associated with the highest number of unique reads, and the taxa with the next highest number of unique reads. In a particular example, a homogeneity metric H can be calculated using the expression

$$H = \frac{{R}^{m}}{{R}^{m} + {R}^{n}} ,$$

where R^{m} is the count of unique reads for the most abundant taxa in a group of taxons that are under a common member of a next highest taxonomic level (e.g., strains of a species or subspecies, subspecies of a species, species of a genus, etc.), and Rⁿ is the count of unique reads for the next most abundant taxa in the same group. In the example shown in FIG. 8, the bottom taxa is the most abundant, and the top is the next most. This homogeneity metric H can be indicative of how much the most abundant taxa dominates at a given taxonomic level. In this formulation, an H=1 can indicate that only a single taxa has a unique read at the taxonomic level for which the calculation is performed. The minimum value for H in this formulation is H=0.5, for example, if there are two taxa that have the count of unique reads (e.g., R^{m}=Rⁿ such that

$$H = \frac{{R}^{m}}{{R}^{m} + {R}^{m}} = 0.5 ,$$

and as R^{m} increases relative to Rⁿ, the value of H also increases).

Note that this is an example, and other homogeneity metric formulations can be used. For example, the most abundant count of unique reads can be evaluated against the total number of unique reads at the taxonomic level being evaluated (e.g.,

$$H = \frac{{R}^{m}}{Σ {R}^{i}} ,$$

where Rⁱ is count of reads for a taxa within the taxonomic level). As another example, the most abundant count of unique reads can be evaluated against the total number of unique reads within the taxonomic level and unique reads at the next higher taxonomic level

$$( H = \frac{{R}^{m}}{Σ {R}^{i} + Σ {R}^{j}} ,$$

where Rⁱ is a count of unique reads for a taxa within the taxonomic level, and R^{j} is a count of unique reads for a taxa within a next higher taxonomic level).

In some embodiments, a uniqueness metric can be calculated based on a homogeneity metric, and the ratio of a count of unique reads associated with a particular taxa and the count of unique reads associated with the most abundant taxa. For example, a uniqueness metric U can be calculated using the expression

$${U}^{i} = H \frac{{R}^{i}}{{R}^{m}} ,$$

where H is a homogeneity metric, R^{m} is the count of unique reads for the most abundant taxa, and Rⁱ is the count of unique reads of the taxa for which the uniqueness metric is being calculated. In the example shown in FIG. 8, as the bottom taxa is the most abundant, if

$$H = \frac{{R}^{m}}{{R}^{m} + {R}^{n}} , {U}^{m} = H \frac{{R}^{m}}{{R}^{m}} = H .$$

Considering the example results shown in FIG. 8, and using

$$H = \frac{{R}^{m}}{{R}^{m} + {R}^{n}} ,$$

H(Taxon 1)=10/(10+10)=0.5, U[A,B]=[0.5,0.5]; H(Taxon 2)=0; H(Taxon 3)=27/(27+1)=0.964, U[E, F, G]=[0.964, 0, 0.36]. In some embodiments, such a uniqueness metric can be used to evaluate a likelihood that a positive is a true positive. For example, higher uniqueness values can be associated with a higher probability of a true positive, while lower uniqueness values can be associated with a lower probability of a true positive.

In some embodiments, when calculating a homogeneity score and/or a uniqueness score at a taxonomic level at which one or more of the members (e.g., taxons) within the taxonomic level that encompasses multiple lower taxons (e.g., at the species/sub-species level in FIG. 8), unique reads associated with the lower taxons encompassed by the member of the taxonomic level can be attributed to the member for the purposes of determining a homogeneity metric for the taxonomic level and/or for calculating a uniqueness metric associated with the member of the taxonomic level. In such embodiments, considering the example results shown in FIG. 8, and using

$$H = \frac{{R}^{m}}{{R}^{m} + {R}^{n}} ,$$

for species/sub-species that share a common higher taxon (e.g., a common genus), which is all species/sub-species in FIG. 8,

$$H ( Genus ) = \frac{{R}^{Taxon 3}}{{R}^{Taxon 3} + {R}^{Taxon 2}} = \frac{28}{28 + 21} = 0.57 ,$$

and U[Taxon1,Taxon2,Taxon3]=[0.42, 0.08, 0.57]. Alternatively, in some embodiments, when calculating a homogeneity score and/or a uniqueness score at a taxonomic level at which one or more of the members (e.g., taxons) within the taxonomic level that encompasses multiple lower taxons (e.g., at the species/sub-species level in FIG. 8), only unique reads associated with that taxonomic level can be considered for the purposes of determining a homogeneity metric for the taxonomic level and/or for calculating a uniqueness metric associated with the member of the taxonomic level. In such embodiments, considering the example results shown in FIG. 8, and using

$$H = \frac{{R}^{m}}{{R}^{m} + {R}^{n}} ,$$

for species/sub-species that share a common higher taxon (e.g., a common genus), which is all species/sub-species in FIG. 8,

$$H = \frac{4}{4 + 1} = 0.8 ,$$

### and U[Taxon1,Taxon2,Taxon3]=[0.2,0.8,0].

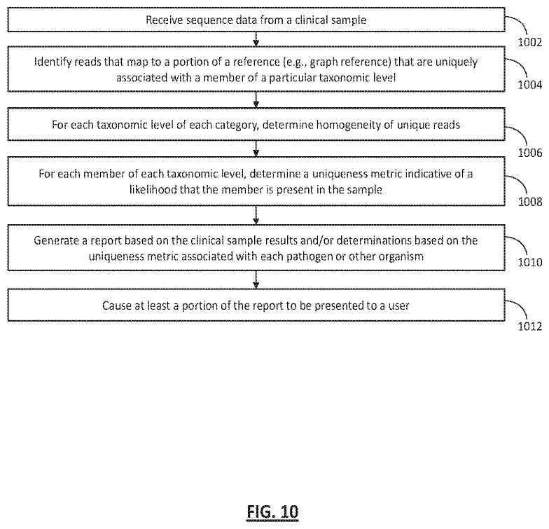

FIG. 10 shows an example 1000 of a process for determining and using a uniqueness metric to classify genetic sequencing results in accordance with some embodiments of the disclosed subject matter.

At 1002, process 1000 can receive genetic data associated with a clinical sample (e.g., from data source 102, from alignment system 106). In some embodiments, the genetic data can be formatted in any suitable format. For example, the genetic data received at 1002 can be formatted in a format described above.

At 1004, process 1000 can identify reads that map to a portion of a reference (e.g., a graph reference) that are uniquely associated with a member (e.g., taxa) of a particular taxonomic level. In some embodiments, process 1000 can use any suitable technique or combination of technique to identify reads that are associated with a single member of a taxonomic level. For example, process 1000 can identify unique reads based on a mapping of each read to one or more reference genomes, and identifying information associated with each portion of the reference genome that the read matches (e.g., matches exactly, matches with gaps, etc.). For example, process 1000 can identify reads that are associated with only a single portion of a reference genome (e.g., a single strain, a single species, etc.) as a unique read for that portion of that reference genome. As another example, In some embodiments, process 1000 can identify reads that are associated with multiple portions of a reference genome that are all encompassed by a single member of a next highest taxonomic level as a unique read for the member of the higher taxonomic level (e.g., a read that matches only strains C and D in FIG. 8 can be identified as a unique read for Taxon2, while a read that matches strains A, B, C and D in FIG. 8 can be identified as a unique read for the genus encompassing Taxons 1, 2, and 3).

At 1006, for each taxonomic level and for each reference (e.g., each genus), process 1000 can determine a homogeneity of the unique reads for that taxonomic level (e.g., a homogeneity metric H). In some embodiments, process 1000 can use any suitable technique or combination of techniques to determine a homogeneity of the unique reads associated with members of a particular taxonomic level (e.g., unique reads associated with strains at a strain level). For example, process 1000 can calculate a homogeneity metric H using any suitable formulation (e.g., as described above in connection with FIG. 9).

At 1008, for each member of each taxonomic level, determine a uniqueness metric indicative of a likelihood that the member is present in the sample (e.g., a uniqueness metric U). In some embodiments, process 1000 can use any suitable technique or combination of techniques to determine a uniqueness metric of unique reads associated with members of a particular taxonomic level (e.g., unique reads associated with strains at a strain level). For example, process 1000 can calculate a uniqueness metric U using any suitable formulation (e.g., as described above in connection with FIG. 9).

At 1010, process 1000 can generate a report based on the clinical sample results and/or determinations based on the uniqueness metric associated with pathogens and/or organisms for which reads were found in the sequence data.

For example, at 1010, process 1000 can use the uniqueness metric and a uniqueness threshold (e.g., set by a user of computing device 110) to determine, for each pathogen represented in the clinical sample results (and/or each pathogen of interest), whether the result is likely clinically significant. For example, if the uniqueness threshold is set to U^{thresh}, process 1000 can determine whether the clinical results for a particular pathogen i meet or exceed the uniqueness threshold based on whether Uⁱ≥U^{thresh}. In such an example, process 1000 can place pathogens that exceed the uniqueness threshold in a more prominent position within a report. Alternatively, in some embodiments, process 1000 can inhibit pathogens that do not exceed the uniqueness threshold from being included in a report. In some embodiments, a uniqueness threshold can be set at any suitable level. A higher threshold can generally be expected to increase precision (e.g., reducing the number of false positives that would have been identified as clinically significant compared to if the uniqueness threshold were not used), and can generally be expected to decrease specificity (e.g., increasing the number of false negatives that would have been otherwise been correctly identified as clinically significant compared to if the uniqueness threshold were not used).

As another example, process 1000 can cause a uniqueness metric associated with a pathogen to be presented in connection with the pathogen, which can add context to results. For example, a clinician can use the uniqueness metric to evaluate whether the presence of reads corresponding to a particular pathogen are likely to be a true positive or a false positive.

In some embodiments, the report can include any suitable content, information, and/or data. For example, the report can include a list of pathogens (if any) that are likely to be clinically significant. As another example, the report can include information indicating confidence in the classification of any positive results (e.g., the uniqueness metric can be indicative of confidence). As yet another example, the report can include graphics (e.g., one or more heatmaps, one or more boxplots, etc.) indicative of the results generated for the clinical sample and/or one or more control samples. As still another example, the report can include a list of pathogens that are unlikely to be clinically significant and/or a list of pathogens for which clinical significance is unclear.

At 1012, process 1000 can cause at least a portion of the report to be presented to a user. For example, in some embodiments, process 1000 can cause a computing device (e.g., computing device 110) to present at least a portion of the report to a user. In some embodiments, process 1000 can cause the report or a portion thereof to be presented in response to a request. As another example, process 1000 can cause the report to be sent to an inbox (e.g., an email account, a healthcare messaging platform, and/or a cellular text messaging service) or other storage location from which the report can be retrieved (e.g., for analysis by a user).

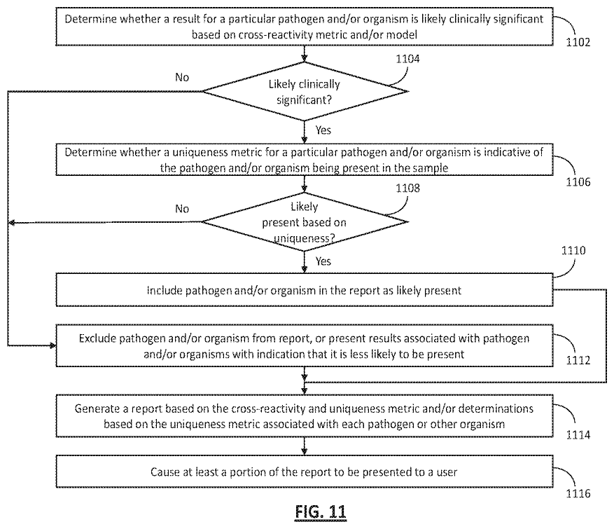

FIG. 11 shows an example 1100 of a process for using pathogen-specific adaptive thresholds and a uniqueness metric to classify genetic sequencing results in accordance with some embodiments of the disclosed subject matter.

At 1102, process 1100 can determine whether a result for a particular pathogen and/or organism is likely clinically significant based on a cross-reactivity metric and/or model. In some embodiments, process 1100 can use any suitable technique or combination of technique to determine whether a result for a particular pathogen and/or organism is likely clinically significant. For example, process 1100 can use techniques described above in connection with FIGS. 3-7 to determine whether a particular result is clinically significant.

At 1104, if process 1100 determines that a result is clinically significant based on cross-reactivity (“YES” at 1104), process 1100 can move to 1106.

At 1106, process 1100 can determine whether a uniqueness metric for a particular pathogen and/or organism is indicative of the pathogen and/or organism being present in the sample. In some embodiments, process 1100 can use any suitable technique or combination of technique to determine whether a result for a particular pathogen and/or organism is likely present based on a uniqueness metric. For example, process 1100 can use techniques described above in connection with FIGS. 8-10 to determine whether a particular result is clinically significant.

At 1108, if process 1100 determines that a pathogen is likely to be present based on uniqueness (“YES” at 1108), process 1100 can move to 1110.

At 1110, process 1100 can include a pathogen and/or organism in a report as likely present in the sample based on the determination at 1102 that the result is likely clinically significant and based on the determination at 1106 that the pathogen is likely present based on unique reads associated with the pathogen.

Otherwise, if process 1100 determines that a result is not clinically significant based on cross-reactivity (“NO” at 1104) and/or if process 1100 determines that a pathogen is unlikely to be present based on uniqueness (“NO” at 1108), process 1100 can move to 1112.

At 1112, process 1100 can exclude the pathogen and/or organism from being included in a report. Alternatively, process 1100 can cause the results associated with the pathogen and/or organism to be presented with an indication that the pathogen and/or organism is less likely to be present (e.g., using an indication that the detected level falls below an LOD, using an indication that the uniqueness metric is below a uniqueness threshold, by placing the pathogen and/or organism in a less prominent portion of the report, etc.).

At 1114, process 1100 can generate a report based on the clinical sample results and/or determinations based on the cross-reactivity and uniqueness metric associated with pathogens and/or organisms for which reads were found in the sequence data.

For example, at 1114, process 1100 can use the indications generated at 1110 and/or 1112 to determine, for each pathogen represented in the clinical sample results (and/or each pathogen of interest), whether the result is likely clinically significant.

As another example, process 1100 can cause information based on cross-reactivity and a uniqueness metric associated with a pathogen to be presented in connection with the pathogen, which can add context to results. For example, a clinician can use the information based on cross-reactivity (e.g., LOD) and uniqueness metric to evaluate whether the presence of reads corresponding to a particular pathogen are likely to be a true positive or a false positive.

In some embodiments, the report can include any suitable content, information, and/or data. For example, the report can include a list of pathogens (if any) that are likely to be clinically significant. As another example, the report can include information indicating confidence in the classification of any positive results (e.g., the information based on cross-reactivity and the uniqueness metric can be indicative of confidence). As yet another example, the report can include graphics (e.g., one or more heatmaps, one or more boxplots, etc.) indicative of the results generated for the clinical sample and/or one or more control samples. As still another example, the report can include a list of pathogens that are unlikely to be clinically significant and/or a list of pathogens for which clinical significance is unclear.

At 1116, process 1100 can cause at least a portion of the report to be presented to a user. For example, in some embodiments, process 1100 can cause a computing device (e.g., computing device 110) to present at least a portion of the report to a user. In some embodiments, process 1100 can cause the report or a portion thereof to be presented in response to a request. As another example, process 1100 can cause the report to be sent to an inbox (e.g., an email account, a healthcare messaging platform, and/or a cellular text messaging service) or other storage location from which the report can be retrieved (e.g., for analysis by a user).

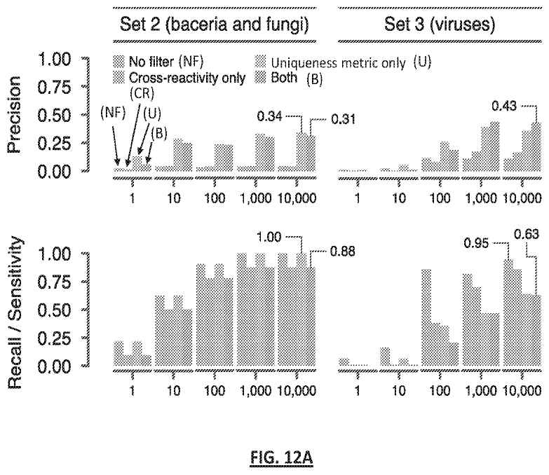

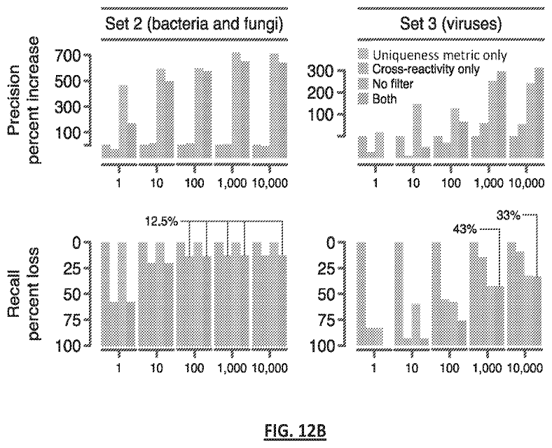

FIGS. 12A and 12B show examples of how using pathogen-specific adaptive thresholds and/or a uniqueness metric can impact the precision and sensitivity of classification of genetic sequencing results in accordance with some embodiments of the disclosed subject matter. FIG. 12A shows precision (TP/(TP+FP) and sensitivity (TP/(TP+FN) for two sets of genetic sequence results generated from samples with various spike-in levels of different bacteria and fungi (set 2) and viruses (set 3), and FIG. 12B shows a difference in precision (as a percent increase) and a decrease in sensitivity (with the axis inverted, such that no increase is shown with a full bar, and a complete loss of sensitivity would be shown with no bar). In the example of FIGS. 12A and 12B, a metric based on the total number of reads that match to a particular member of a taxonomic level (e.g., a particular strain) can be used to determine whether a particular pathogen and/or organisms is present in the “no filter” results. The uniqueness threshold used for both the uniqueness only and both results was U≥0.3, such that if U was greater than or equal to 0.3 the result was considered a positive for that pathogen, while if U was less than 0.3 the result was considered a negative for that pathogen. As shown in FIGS. 12A and 12B, using techniques described herein based on cross-reactivity generally increased precision by a relatively small amount for set 2, and resulted in increased precision for set 3 at higher spike-in levels, while having a relatively small impact on sensitivity for set 2 and for set 3 at higher spike-in levels. Using techniques described herein based on uniqueness of read mappings, generally increased precision by large amounts (e.g., multiple orders of magnitude) for set 2 and for set 3 at most spike-in levels, while having no impact on sensitivity for set 2, and a relatively large impact on sensitivity for set 3. The combination of techniques described herein based on cross-reactivity and techniques described herein based on uniqueness of read mappings had slightly lower precision and sensitivity for set 2 (which is likely due to E. coli, which was difficult to call in the example sample due to a relatively large LOD calculated for E. coli (e.g., based on a high degree of cross-reactivity between E. coli strains) Additionally, note that there were many closely related organisms in Set 3, which likely increased the number of false negatives caused by the uniqueness metric. For example, the presence of closely related organisms can lead to the needle-in-a-haystack assumption being incorrect, and thus unique reads may be correctly identified for many related organisms, which can result in H being reduced relative to a sample in which fewer closely related organisms, which can in turn lead to reduced U for those organisms.

### Further Examples Having a Variety of Features:

Implementation examples are described in the following numbered clauses:

1. A method for classifying a genetic sequencing result for a sample, the method comprising: receiving a clinical sample genetic sequencing result for a clinical sample; the clinical sample genetic sequencing result comprising a plurality of values that are each indicative of a number of reads detected in the clinical sample for a respective reference organism of a plurality of reference organisms; identifying a value in the clinical sample genetic sequencing result that is over a detection threshold associated with an organism; determining, utilizing a model, that the value is unlikely to be diagnostically significant; generating a report based on the clinical sample genetic sequencing result and any reference organisms associated with a value identified as likely to be diagnostically significant; and causing at least a portion of the report to be presented to a user with an indication of any organisms associated with a value identified as likely to be diagnostically significant.

2. The method of clause 1, further comprising: receiving a plurality of sample genetic sequencing results for a plurality of reference organisms corresponding to a respective plurality of reference organism samples; generating a distribution for each of reference organisms in the plurality of reference organisms based on the plurality of sample genetic sequencing results; associating, for each of the plurality of reference organisms, a threshold that is based on the distribution; generating at least one matrix of replicate-averaged signal for each reference organism in the plurality of reference organisms by cross-referencing at least one synthetic genetic sequencing result for each reference organism with at least one other synthetic genetic sequencing result for said same reference organism; updating the threshold for each reference organism based on the matrix of replicate-averaged signal; and identifying, utilizing the model, any values in the clinical sample genetic sequencing result that are likely to be diagnostically significant based on the threshold associated with each reference organism.

3. The method of clause 2, further comprising setting the threshold for each of the plurality of reference organisms at the median of the distribution associated with that reference organism.

4. The method of any one of clauses 1 to 3, further comprising: training a neural network using the plurality of synthetic genetic sequencing results; providing the clinical sample genetic sequencing result as input to the trained neural network; and receiving, from the trained neural network, output identifying any values in the clinical sample genetic sequencing result that are likely to be diagnostically significant.

5. The method of any one of clauses 1 to 4, further comprising: receiving at least one sample genetic sequencing result for a reference organism corresponding to a respective reference organism sample; receiving at least one sample genetic sequencing result for a host organism corresponding to a respective host organism sample; generating a plurality of synthetic genetic sequencing results corresponding to a respective plurality of synthetic samples each containing a combination of the host reference organism and the reference organism by combining at least a portion of the sample genetic sequencing result for the reference organism with at least a portion of the sample genetic sequencing result for the host organism for each synthetic sample, each synthetic genetic sequencing result comprising a plurality of values that are each indicative of a number of reads detected in the synthetic sample for a respective reference organism; generating at least one matrix of replicate-averaged signal by cross-referencing at least one synthetic genetic sequencing result with at least one other synthetic genetic sequencing result; generating a model based on the at least one sample genetic sequencing result for a reference organism and the at least one sample genetic sequencing result for a host organism; determining at least one threshold based on the at least one matrix of replicate-averaged signal; and updating at least a portion of the model based on the at least one threshold.

6. The method of any one of clauses 1 to 5, further comprising: (i) receiving a plurality of sample genetic sequencing results for a plurality of reference organisms corresponding to a respective plurality of reference organism samples; (ii) generating a synthetic genetic sequencing result by combining at least a portion of a sample genetic sequencing result for a reference organism with at least a portion of the sample genetic sequencing result for the host organism; and (iii) repeating (ii) for each reference organism sample of the plurality of reference organism samples.

7. The method of clause 6, further comprising: generating a sufficient number of synthetic genetic sequencing results such that the number of synthetic genetic sequencing results in the plurality of synthetic genetic sequencing results is at least 10× greater than the number of sample genetic sequencing results for reference organisms in the plurality of sample genetic sequencing results for a plurality of reference organisms.

8. The method of any one of clauses 1 to 7, further comprising: determining at least one threshold based on the at least one matrix of replicate-averaged signal, using a combination of conditional probability and at least one loss function.

9. The method of clause 8, further comprising: determining at least one threshold based on the at least one matrix of replicate-averaged signal, using a combination of conditional probability and at least one loss function.

10. A method for classifying a genetic sequencing result for a sample, the method comprising: receiving a clinical sample genetic sequencing result for a clinical sample, the clinical sample genetic sequencing result comprising a plurality of values that are each indicative of a number of reads detected in the clinical sample for a respective reference organism of a plurality of reference organisms; identifying, for each of a plurality of members of a taxonomic level, a count of unique reads that align with only a single member of that taxonomic level; determining, for the taxonomic level, a homogeneity metric H indicative how high the unique read count of a member of the taxonomic level with a highest unique read count is compared to a member of the taxonomic level with a next highest unique read count; determining, for each of the plurality of members of the taxonomic level, a uniqueness metric U based on the count of unique reads associated with that member and the homogeneity metric associated with the taxonomic level; generating a report based on the clinical sample genetic sequencing result and the uniqueness metric associated with each of the plurality of members of the taxonomic level; and causing at least a portion of the report to be presented to a user with an indication of any organisms associated with a uniqueness value above a threshold.

11. The method of clause 10, wherein the plurality of members the taxonomic level correspond to different strains.

12. The method of any one of clauses 10 or 11, wherein the plurality of members the taxonomic level correspond to different species.

13. The method of any one of clauses 10 to 12, wherein the homogeneity metric is calculated using the following:

$$H = \frac{{R}^{m}}{{R}^{m} + {R}^{n}} ,$$

where R^{m} is the count of unique reads for the member with the highest count of unique reads, and Rⁿ is the count of unique reads for the member with the next highest count of unique reads.

14. The method of any one of clauses 10 to 13, wherein the uniqueness metric is calculated using the following:

$$U = H \frac{{R}^{i}}{{R}^{m}} ,$$

where R^{m} is the count of unique reads for the member with the highest count of unique reads, and Rⁱ is the count of unique reads of the member for which U is being determined.

15. The method of any one of clauses 10 to 14, further comprising: identifying, for each of a plurality of members of a taxonomic level, the count of unique reads.

16. A method for classifying a genetic sequencing result for a sample, the method comprising: receiving a clinical sample genetic sequencing result for a clinical sample, the clinical sample genetic sequencing result comprising a plurality of values that are each indicative of a number of reads detected in the clinical sample for a respective reference organism of a plurality of reference organisms; identifying a value in the clinical sample genetic sequencing result that is over a detection threshold associated with a member of a taxonomic level; determining, utilizing a model, that the value is unlikely to be diagnostically significant; identifying, for each of a plurality of members of the taxonomic level, a count of unique reads that align with only a single member of that taxonomic level; determining, for the taxonomic level, a homogeneity metric H indicative how high the unique read count of a member of the taxonomic level with a highest unique read count is compared to a member of the taxonomic level with a next highest unique read count; determining, for each of the plurality of members of the taxonomic level, a uniqueness metric U based on the count of unique reads associated with that member and the homogeneity metric associated with the taxonomic level; generating a report based on the clinical sample genetic sequencing result, the uniqueness metric associated with each of the plurality of members of the taxonomic level, and any reference organisms associated with a value identified as likely to be diagnostically significant; and causing at least a portion of the report to be presented to a user with an indication of any organisms associated with a value identified as likely to be diagnostically significant.

17. A non-transitory computer-readable medium storing computer-executable code, comprising code for causing a computer to cause a processor to: perform a method of any of clauses 1 to 16.

18. A system, comprising: at least one processor that is configured to: perform a method of any of clauses 1 to 16.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as RAM, Flash memory, electrically programmable read only memory (EPROM), electrically erasable programmable read only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

It should be noted that, as used herein, the term mechanism can encompass hardware, software, firmware, or any suitable combination thereof.

It should be understood that the above described steps of the processes of FIGS. 3, 4, 10, and 11 can be executed or performed in any order or sequence not limited to the order and sequence shown and described in the figures. Also, some of the above steps of the processes of FIGS. 3, 4, 10 and/or 11 can be executed or performed substantially simultaneously where appropriate or in parallel to reduce latency and processing times.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways.

1. A system for classifying a genetic sequencing result for a sample, the system comprising:
at least one hardware processor that is programmed to:
receive a clinical sample genetic sequencing result for a clinical sample,
the clinical sample genetic sequencing result comprising a plurality of values that are each indicative of a number of reads detected in the clinical sample for a respective reference organism of a plurality of reference organisms;
identify a value in the clinical sample genetic sequencing result that is over a detection threshold associated with an organism;
determine, utilizing a model, that the value is unlikely to be diagnostically significant;
generate a report based on the clinical sample genetic sequencing result and any reference organisms associated with a value identified as likely to be diagnostically significant; and
cause at least a portion of the report to be presented to a user with an indication of any organisms associated with a value identified as likely to be diagnostically significant.

2. The system of claim 1, wherein the at least one hardware processor is further programmed to:
receive a plurality of sample genetic sequencing results for a plurality of reference organisms corresponding to a respective plurality of reference organism samples;
generate a distribution for each of reference organisms in the plurality of reference organisms based on the plurality of sample genetic sequencing results;
associate, for each of the plurality of reference organisms, a threshold that is based on the distribution; and
generate at least one matrix of replicate-averaged signal for each reference organism in the plurality of reference organisms by cross-referencing at least one synthetic genetic sequencing result for each reference organism with at least one other synthetic genetic sequencing result for said same reference organism;
update the threshold for each reference organism based on the matrix of replicate-averaged signal;
identify, utilizing the model, any values in the clinical sample genetic sequencing result that are likely to be diagnostically significant based on the threshold associated with each reference organism.

3. The system of claim 2, wherein the at least one hardware processor is further programmed to set the threshold for each of the plurality of reference organisms at the median of the distribution associated with that organism.

4. The system of claim 1, wherein the at least one hardware processor is further programmed to:
train a neural network using the plurality of synthetic genetic sequencing results;
provide the clinical sample genetic sequencing result as input to the trained neural network; and
receive, from the trained neural network, output identifying any values in the clinical sample genetic sequencing result that are likely to be diagnostically significant.

5. The system of claim 4, wherein the at least one hardware processor is further programmed to:
receive at least one sample genetic sequencing result for a reference organism corresponding to a respective reference organism sample;
receive at least one sample genetic sequencing result for a host organism corresponding to a respective host organism sample;
generate a plurality of synthetic genetic sequencing results corresponding to a respective plurality of synthetic samples each containing a combination of the host reference organism and the reference organism by combining at least a portion of the sample genetic sequencing result for the reference organism with at least a portion of the sample genetic sequencing result for the host organism for each synthetic sample,
each synthetic genetic sequencing result comprising a plurality of values that are each indicative of a number of reads detected in the synthetic sample for a respective reference organism;
generate at least one matrix of replicate-averaged signal by cross-referencing at least one synthetic genetic sequencing result with at least one other synthetic genetic sequencing result;
generate a model based on the at least one sample genetic sequencing result for a reference organism and the at least one sample genetic sequencing result for a host organism;
determine at least one threshold based on the at least one matrix of replicate-averaged signal; and
update at least a portion of the model based on the at least one threshold.

6. The system of claim 1, wherein the at least one hardware processor is further programmed to:
(i) receive a plurality of sample genetic sequencing results for a plurality of reference organisms corresponding to a respective plurality of reference organism samples;
(ii) generate a synthetic genetic sequencing result by combining at least a portion of a sample genetic sequencing result for a reference organism with at least a portion of the sample genetic sequencing result for the host organism; and
(iii) repeat (ii) for each reference organism sample of the plurality of reference organism samples.

7. The system of claim 6, wherein the at least one hardware processor is further programmed to:
generate a sufficient number of synthetic genetic sequencing results such that the number of synthetic genetic sequencing results in the plurality of synthetic genetic sequencing results is at least 10× greater than the number of sample genetic sequencing results for reference organisms in the plurality of sample genetic sequencing results for a plurality of reference organisms.

8. The system of claim 1, wherein the at least one hardware processor is further programmed to:
determine at least one threshold based on the at least one matrix of replicate-averaged signal, using conditional probability.

9. The system of claim 8, wherein the at least one hardware processor is further programmed to:
determine at least one threshold based on the at least one matrix of replicate-averaged signal, using a combination of conditional probability and at least one loss function.

10. A method for classifying a genetic sequencing result for a sample, the method comprising:
receiving a clinical sample genetic sequencing result for a clinical sample;
the clinical sample genetic sequencing result comprising a plurality of values that are each indicative of a number of reads detected in the clinical sample for a respective reference organism of a plurality of reference organisms;
identifying a value in the clinical sample genetic sequencing result that is over a detection threshold associated with an organism;
determining, utilizing a model, that the value is unlikely to be diagnostically significant;
generating a report based on the clinical sample genetic sequencing result and any reference organisms associated with a value identified as likely to be diagnostically significant; and
causing at least a portion of the report to be presented to a user with an indication of any organisms associated with a value identified as likely to be diagnostically significant.

11. The method of claim 10, further comprising:
receiving a plurality of sample genetic sequencing results for a plurality of reference organisms corresponding to a respective plurality of reference organism samples;
generating a distribution for each of reference organisms in the plurality of reference organisms based on the plurality of sample genetic sequencing results;
associating, for each of the plurality of reference organisms, a threshold that is based on the distribution;
generating at least one matrix of replicate-averaged signal for each reference organism in the plurality of reference organisms by cross-referencing at least one synthetic genetic sequencing result for each reference organism with at least one other synthetic genetic sequencing result for said same reference organism;
updating the threshold for each reference organism based on the matrix of replicate-averaged signal; and
identifying, utilizing the model, any values in the clinical sample genetic sequencing result that are likely to be diagnostically significant based on the threshold associated with each reference organism.

12. The method of claim 11, further comprising setting the threshold for each of the plurality of reference organisms at the median of the distribution associated with that reference organism.

13. The method of claim 10, further comprising:
training a neural network using the plurality of synthetic genetic sequencing results;
providing the clinical sample genetic sequencing result as input to the trained neural network; and
receiving, from the trained neural network, output identifying any values in the clinical sample genetic sequencing result that are likely to be diagnostically significant.

14. The method of claim 10, further comprising:
receiving at least one sample genetic sequencing result for a reference organism corresponding to a respective reference organism sample;
receiving at least one sample genetic sequencing result for a host organism corresponding to a respective host organism sample;
generating a plurality of synthetic genetic sequencing results corresponding to a respective plurality of synthetic samples each containing a combination of the host reference organism and the reference organism by combining at least a portion of the sample genetic sequencing result for the reference organism with at least a portion of the sample genetic sequencing result for the host organism for each synthetic sample,
each synthetic genetic sequencing result comprising a plurality of values that are each indicative of a number of reads detected in the synthetic sample for a respective reference organism;
generating at least one matrix of replicate-averaged signal by cross-referencing at least one synthetic genetic sequencing result with at least one other synthetic genetic sequencing result;
generating a model based on the at least one sample genetic sequencing result for a reference organism and the at least one sample genetic sequencing result for a host organism;
determining at least one threshold based on the at least one matrix of replicate-averaged signal; and
updating at least a portion of the model based on the at least one threshold.

15. The method of claim 10, further comprising:
(i) receiving a plurality of sample genetic sequencing results for a plurality of reference organisms corresponding to a respective plurality of reference organism samples;
(ii) generating a synthetic genetic sequencing result by combining at least a portion of a sample genetic sequencing result for a reference organism with at least a portion of the sample genetic sequencing result for the host organism; and
(iii) repeating (ii) for each reference organism sample of the plurality of reference organism samples.

16. The method of claim 10, further comprising:
determining at least one threshold based on the at least one matrix of replicate-averaged signal, using a combination of conditional probability and at least one loss function.

17. A non-transitory computer readable medium containing computer executable instructions that, when executed by a processor, cause the processor to perform a method for classifying a genetic sequencing result for a sample, the method comprising:
receiving a clinical sample genetic sequencing result for a clinical sample,
the clinical sample genetic sequencing result comprising a plurality of values that are each indicative of a number of reads detected in the clinical sample for a respective reference organism of a plurality of reference organisms;
identifying a value in the clinical sample genetic sequencing result that is over a detection threshold associated with an organism;
determining, utilizing a model, that the value is unlikely to be diagnostically significant;
generating a report based on the clinical sample genetic sequencing result and any reference organisms associated with a value identified as likely to be diagnostically significant; and
causing at least a portion of the report to be presented to a user with an indication of any organisms associated with a value identified as likely to be diagnostically significant.

18. The non-transitory computer readable medium of claim 17, wherein the method further comprises:
receiving a plurality of sample genetic sequencing results for a plurality of reference organisms corresponding to a respective plurality of reference organism samples;
generating a distribution for each of reference organisms in the plurality of reference organisms based on the plurality of sample genetic sequencing results;
associating, for each of the plurality of reference organisms, a threshold that is based on the distribution;
generating at least one matrix of replicate-averaged signal for each reference organism in the plurality of reference organisms by cross-referencing at least one synthetic genetic sequencing result for each reference organism with at least one other synthetic genetic sequencing result for said same reference organism;
updating the threshold for each reference organism based on the matrix of replicate-averaged signal; and
identifying, utilizing the model, any values in the clinical sample genetic sequencing result that are likely to be diagnostically significant based on the threshold associated with each reference organism.

19. The non-transitory computer readable medium of claim 17, wherein the method further comprises:
receiving at least one sample genetic sequencing result for a reference organism corresponding to a respective reference organism sample;
receiving at least one sample genetic sequencing result for a host organism corresponding to a respective host organism sample;
generating a plurality of synthetic genetic sequencing results corresponding to a respective plurality of synthetic samples each containing a combination of the host reference organism and the reference organism by combining at least a portion of the sample genetic sequencing result for the reference organism with at least a portion of the sample genetic sequencing result for the host organism for each synthetic sample,
each synthetic genetic sequencing result comprising a plurality of values that are each indicative of a number of reads detected in the synthetic sample for a respective reference organism;
generating at least one matrix of replicate-averaged signal by cross-referencing at least one synthetic genetic sequencing result with at least one other synthetic genetic sequencing result;
generating a model based on the at least one sample genetic sequencing result for a reference organism and the at least one sample genetic sequencing result for a host organism;
determining at least one threshold based on the at least one matrix of replicate-averaged signal; and
updating at least a portion of the model based on the at least one threshold.

20. The non-transitory computer readable medium of claim 17, wherein the method further comprises:
determining at least one threshold based on the at least one matrix of replicate-averaged signal, using at a combination of conditional probability and at least one loss function.

21-41. (canceled)
